SEQUENCE LISTING (49 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 134 aa, Artificial Sequence
  Synthetic Construct
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - DNA, 405 bp, Artificial Sequence
  Synthetic Construct
atggcgcctacatccagctcgaccaaaaagacgcagctgcaactggaacacctgctcctggatctgcaaatgattcttaacggtatcaataactacaaaaatccgaaactgacccgtatgctgacgtttaaattctatatgccaaagaaagcgaccgagctgaaacatctgcagtgcctggaagaggaactgaaaccgctggaggaagttttgaacctggctcagtctaaaaactttcacctgcgccctcgtgacctgatttccaatatcaacgtgattgttctggaactgaaaggctctgaaaccacgtttatgtgcgagtacgccgatgaaaccgccacgattgtggaatttctgaatcgctggatcaccttctcccagagcattattagcacgctgacctaa

SEQ ID NO: 5 - DNA, 5398 bp, Artificial Sequence
  Synthetic Construct
ggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttaggtcagcgtgctaataatgctctgggagaaggtgatccagcgattcagaaattccacaatcgtggcggtttcatcggcgtactcgcacataaacgtggtttcagagcctttcagttccagaacaatcacgttgatattggaaatcaggtcacgagggcgcaggtgaaagtttttagactgagccaggttcaaaacttcctccagcggtttcagttcctcttccaggcactgcagatgtttcagctcggtcgctttctttggcatatagaatttaaacgtcagcatacgggtcagtttcggatttttgtagttattgataccgttaagaatcatttgcagatccaggagcaggtgttccagttgcagctgcgtctttttggtcgagctggatgtaggcgccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgaagcttctatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggactgcagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcggttcagggcagggtcgttaaatagccgcttatgtctattgctggtttaccggtttattgactaccggaagcagtgtgaccgt

SEQ ID NO: 6 - DNA, 35 bp, Artificial Sequence
  Synthetic Construct
caataactacaaaaattagaaactgacccgtatgc

SEQ ID NO: 7 - DNA, 35 bp, Artificial Sequence
  Synthetic Construct
gcatacgggtcagtttctaatttttgtagttattg

SEQ ID NO: 8 - DNA, 35 bp, Artificial Sequence
  Synthetic Construct
taactacaaaaatccgtagctgacccgtatgctga

SEQ ID NO: 9 - DNA, 35 bp, Artificial Sequence
  Synthetic Construct
tcagcatacgggtcagctacggatttttgtagtta

SEQ ID NO: 10 - DNA, 35 bp, Artificial Sequence
  Synthetic Construct
caaaaatccgaaactgtagcgtatgctgacgttta

SEQ ID NO: 11 - DNA, 35 bp, Artificial Sequence
  Synthetic Construct
taaacgtcagcatacgctacagtttcggatttttg

SEQ ID NO: 12 - DNA, 35 bp, Artificial Sequence
  Synthetic Construct
aaatccgaaactgacctagatgctgacgtttaaat

SEQ ID NO: 13 - DNA, 35 bp, Artificial Sequence
  Synthetic Construct
atttaaacgtcagcatctaggtcagtttcggattt

SEQ ID NO: 14 - DNA, 35 bp, Artificial Sequence
  Synthetic Construct
gaaactgacccgtatgtagacgtttaaattctata

SEQ ID NO: 15 - DNA, 35 bp, Artificial Sequence
  Synthetic Construct
tatagaatttaaacgtctacatacgggtcagtttc

SEQ ID NO: 16 - DNA, 35 bp, Artificial Sequence
  Synthetic Construct
actgacccgtatgctgtagtttaaattctatatgc

SEQ ID NO: 17 - DNA, 35 bp, Artificial Sequence
  Synthetic Construct
gcatatagaatttaaactacagcatacgggtcagt

SEQ ID NO: 18 - DNA, 35 bp, Artificial Sequence
  Synthetic Construct
gacccgtatgctgacgtagaaattctatatgccaa

SEQ ID NO: 19 - DNA, 35 bp, Artificial Sequence
  Synthetic Construct
ttggcatatagaatttctacgtcagcatacgggtc

SEQ ID NO: 20 - DNA, 35 bp, Artificial Sequence
  Synthetic Construct
ccgtatgctgacgttttagttctatatgccaaaga

SEQ ID NO: 21 - DNA, 35 bp, Artificial Sequence
  Synthetic Construct
tctttggcatatagaactaaaacgtcagcatacgg

SEQ ID NO: 22 - DNA, 35 bp, Artificial Sequence
  Synthetic Construct
tatgctgacgtttaaatagtatatgccaaagaaag

SEQ ID NO: 23 - DNA, 35 bp, Artificial Sequence
  Synthetic Construct
ctttctttggcatatactatttaaacgtcagcata

SEQ ID NO: 24 - DNA, 35 bp, Artificial Sequence
  Synthetic Construct
gctgacgtttaaattctagatgccaaagaaagcga

SEQ ID NO: 25 - DNA, 35 bp, Artificial Sequence
  Synthetic Construct
tcgctttctttggcatctagaatttaaacgtcagc

SEQ ID NO: 26 - DNA, 35 bp, Artificial Sequence
  Synthetic Construct
tctgcagtgcctggaataggaactgaaaccgctgg

SEQ ID NO: 27 - DNA, 35 bp, Artificial Sequence
  Synthetic Construct
ccagcggtttcagttcctattccaggcactgcaga

SEQ ID NO: 28 - DNA, 35 bp, Artificial Sequence
  Synthetic Construct
gcagtgcctggaagagtagctgaaaccgctggagg

SEQ ID NO: 29 - DNA, 35 bp, Artificial Sequence
  Synthetic Construct
cctccagcggtttcagctactcttccaggcactgc

SEQ ID NO: 30 - DNA, 35 bp, Artificial Sequence
  Synthetic Construct
cctggaagaggaactgtagccgctggaggaagttt

SEQ ID NO: 31 - DNA, 35 bp, Artificial Sequence
  Synthetic Construct
aaacttcctccagcggctacagttcctcttccagg

SEQ ID NO: 32 - DNA, 35 bp, Artificial Sequence
  Synthetic Construct
ggaagaggaactgaaatagctggaggaagttttga

SEQ ID NO: 33 - DNA, 35 bp, Artificial Sequence
  Synthetic Construct
tcaaaacttcctccagctatttcagttcctcttcc

SEQ ID NO: 34 - DNA, 35 bp, Artificial Sequence
  Synthetic Construct
ggaactgaaaccgctgtaggaagttttgaacctgg

SEQ ID NO: 35 - DNA, 35 bp, Artificial Sequence
  Synthetic Construct
ccaggttcaaaacttcctacagcggtttcagttcc

SEQ ID NO: 36 - DNA, 35 bp, Artificial Sequence
  Synthetic Construct
actgaaaccgctggagtaggttttgaacctggctc

SEQ ID NO: 37 - DNA, 35 bp, Artificial Sequence
  Synthetic Construct
gagccaggttcaaaacctactccagcggtttcagt

SEQ ID NO: 38 - DNA, 35 bp, Artificial Sequence
  Synthetic Construct
gctggaggaagttttgtagctggctcagtctaaaa

SEQ ID NO: 39 - DNA, 35 bp, Artificial Sequence
  Synthetic Construct
ttttagactgagccagctacaaaacttcctccagc

SEQ ID NO: 40 - DNA, 35 bp, Artificial Sequence
  Synthetic Construct
ggaggaagttttgaactaggctcagtctaaaaact

SEQ ID NO: 41 - DNA, 35 bp, Artificial Sequence
  Synthetic Construct
agtttttagactgagcctagttcaaaacttcctcc

SEQ ID NO: 42 - DNA, 35 bp, Artificial Sequence
  Synthetic Construct
cacgtttatgtgcgagtaggccgatgaaaccgcca

SEQ ID NO: 43 - DNA, 35 bp, Artificial Sequence
  Synthetic Construct
tggcggtttcatcggcctactcgcacataaacgtg

SEQ ID NO: 44 - DNA, 42 bp, Artificial Sequence
  Synthetic Construct
tgagcggataacaattcccctctagaaataattttgtttaac

SEQ ID NO: 45 - DNA, 31 bp, Artificial Sequence
  Synthetic Construct
gatctcagtggtggtggtggtggtgctcgag

SEQ ID NO: 46 - DNA, 2997 bp, Artificial Sequence
  Synthetic Construct
ccttatgcgactccctgcattagggagctgttgacaattaatcatcggctcgtataatgtgtggaattgtgagcggataacaatttcacaaaggaggtcatatggataaaaagcctctgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaagatctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctggccccgaacctgtacaactatctgcgtaaactggaccgtgccctgccggacccgatcaaaattttcgagatcggtccttgctaccgtaaagagtccgacggtaaagagcacctggaagaattcaccatgctgaacttttgccagatgggtagcggttgcacgcgtgaaaacctggaatccattatcaccgacttcctgaatcacctgggtatcgatttcaaaattgttggtgacagctgtatggtgtacggcgatacgctggatgttatgcacggcgatctggagctgtcttccgcagtagtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaactagtgtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgagcactgcagcggctaactaagcggcctgctgactttctcgccgatcaaaaggcattttgctattaagggattgacgagggcgtatctgcgcagtaagatgcgccccgcattggaaacctgatcatgtagatcgaatggactctaaatccgttcagccgggttagattcccggggtttccgccaaattcgaaaagcctgctcaacgagcaggcttttttgcatctcgagcagctcagggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgcgcgctgcggacac

SEQ ID NO: 47 - DNA, 2153 bp, Artificial Sequence
  Synthetic Construct
cggacatcagcgcgcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactc

SEQ ID NO: 48 - DNA, 35 bp, Artificial Sequence
  Synthetic Construct
gatttccaatatcaactagattgttctggaactga

SEQ ID NO: 49 - DNA, 35 bp, Artificial Sequence
  Synthetic Construct
tcagttccagaacaatctagttgatattggaaatc

(19) United States
(12) Patent Application Publication
(10) Pub. No.: US 2025/0213703 A1
(43) Pub. Date: Jul. 3, 2025

(54) HUMAN INTERLEUKIN 2-POLYETHYLENE GLYCOL CONJUGATE AND USE THEREOF

(71) Applicant: Novocodex Biopharmaceuticals Co., Ltd., Yuecheng District Shaoxing, Zhejiang (CN)

(72) Inventors: Xuejun LIANG, Yuecheng District Shaoxing, Zhejiang (CN); Chenghao YE, Yuecheng District Shaoxing, Zhejiang (CN); Gang XIA, Yuecheng District Shaoxing, Zhejiang (CN); Longfei CHEN, Yuecheng District Shaoxing, Zhejiang (CN); Pengchao HUO, Yuecheng District Shaoxing, Zhejiang (CN); Jinwei YANG, Yuecheng District Shaoxing, Zhejiang (CN); Yuebin YING, Yuecheng District Shaoxing, Zhejiang (CN); Zunyang GONG, Yuecheng District Shaoxing, Zhejiang (CN); Xin HENG, Yuecheng District Shaoxing, Zhejiang (CN); Xiaoyu ZHU, Yuecheng District Shaoxing, Zhejiang (CN); Lifei ZHU, Yuecheng District Shaoxing, Zhejiang (CN); Hao HUANG, Yuecheng District Shaoxing, Zhejiang (CN); Wen DING, Yuecheng District Shaoxing, Zhejiang (CN); Lin JIAO, Yuecheng District Shaoxing, Zhejiang (CN); Jingjing ZHU, Yuecheng District Shaoxing, Zhejiang (CN)

(73) Assignee: Novocodex Biopharmaceuticals Co., Ltd., Yuecheng District Shaoxing, Zhejiang (CN)

(21) Appl. No.: 18/687,683

(22) PCT Filed: Mar. 2, 2022
(86) PCT No.: PCT/CN2022/078827

§ 371 Date: Mar. 18, 2025

(30) Foreign Application Priority Data
Sep. 1, 2021 (CN) 202111019753.3

(51) Int. Cl. A61K 47/60; A61K 38/20; A61K 47/54; A61P 35/00; C12P 21/02

(52) U.S. Cl. CPC A61K 47/60; A61K 38/2013; A61K 47/542; A61P 35/00; C12P 21/02

(57) ABSTRACT

The invention provides a human interleukin 2-polyethylene glycol conjugate and use thereof. The human interleukin 2-polyethylene glycol conjugate provided by the invention comprises a recombinant human interleukin 2 containing at least one unnatural amino acid and PEG coupled to the at least one unnatural amino acid. The unnatural amino acid is a compound containing a carbonyl terminal group and having a structure as shown in formula (I) or an enantiomer thereof, and the PEG is coupled to the at least one unnatural amino acid by forming an oxime bond between the carbonyl terminal group and the PEG containing a hydroxylamine terminal group. The human interleukin 2-polyethylene glycol conjugate provided by the invention can be used individually or in combination with other anti-tumor drugs for the treatment of diseases such as solid tumors and hematologic tumors.

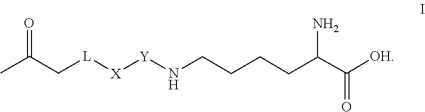

### CROSS-REFERENCE TO RELATED APPLICATIONS

This application is the 371 National Phase of International Application No. PCT/CN2022/078827 filed Mar. 2, 2022, which claims priority to and the benefit of the earlier filing of CN 202111019753.3, filed Sep. 1, 2021, is incorporated by reference herein in its entirety.

### INCORPORATION BY REFERENCE OF SEQUENCE LISTING

A computer readable text file, entitled “S481-6002US_Sub_SeqList.txt” created on or about Dec. 13, 2024, with a file size of 28,672 bytes, contains the sequence listing for this application and is hereby incorporated by reference in its entirety.

### TECHNICAL FIELD

The present invention relates to the field of biopharmaceuticals, and particularly relates to a human interleukin 2-polyethylene glycol conjugate and use thereof.

### BACKGROUND

Interleukin 2 (IL-2) is an important immunoregulatory factor produced from activated type I helper T lymphocytes (Th1), which was once called a T cell growth factor, with a main biological function of promoting the growth, proliferation and differentiation of T cells (comprising CD4⁺ and CD8⁺ T cells) by dual ways of stimulation and anti-apoptosis, and promoting the further secretion of cytokines. In addition, interleukin 2 also stimulates the proliferation of NK cells, enhances the killing activity of NK and produces the cytokines, and induces the production of LAK cells; and promotes the proliferation of B cells and secretion of antibodies. Therefore, interleukin 2 plays an important role in immune response and antiviral infection (Gaffena S. L, Cytokine 28: 109e123, 2004).

IL-2 has been widely used in clinic since it was first discovered by Morgan et al. in 1976. In 1991, rhIL-2 (product name: Aldesleukin) produced by Cetus in the United States was approved by FDA, which was widely used in malignant tumors such as renal cell carcinoma, malignant melanoma and malignant lymphoma (the instruction of Proleukin), and also had potential effects in the adjuvant treatment of hepatitis B and hepatitis C infection (Tomova R. et al., Anticancer Research, 29:5241-5244, 2009). Up to now, more than 10 recombinant human interleukin 2 biological products have been put into production and listed in China, which are widely used in the treatment of malignant tumors such as renal cell carcinoma, melanoma, breast cancer, bladder cancer, liver cancer, rectal cancer, lymphoma and lung cancer, used for the control of cancerous hydrothorax and ascites, used for the enhancement of immune function of tumor patients after surgery, radiotherapy and chemotherapy, used for the improvement of cellular immune function and anti-infection ability of patients with congenital or acquired immunodeficiency, and used for the treatment of various autoimmune diseases such as rheumatoid arthritis, systemic lupus erythematosus, and Sjogren syndrome, and have a certain therapeutic effect on some viral, bacillary and intracellular bacterial parasitic infectious diseases, such as hepatitis B, leprosy, tuberculosis and Candida albicans infection.

The precursor of human IL-2 is composed of 153 amino acid residues, and when the precursor is secreted out of cells, the signal peptide (comprising 20 amino acid residues) of the precursor is excised, and a mature IL-2 with 133 amino acids is produced, with a molecular weight of 15.4 kD. The activating effect of IL-2 on effector cells is realized by binding with an IL-2 receptor (IL-2R) on a cell surface. It has been found that the IL-2 receptor comprises IL-2Rα, IL-2Rβ and IL-2Rγ, which may form a heteromultimeric glycoprotein functional complex IL-2Rαβγ with high-affinity (Kd=10⁻¹¹ mol/L). IL-2Rβ and IL-2Rγ may form a receptor complex IL-2Rβγ with moderate-affinity (Kd=10⁻⁹ mol/L), which has biological activity after being activated by IL-2. IL-2Rα subunit is in a form of low-affinity receptor (Kd=10⁻⁸ mol/L), which cannot transmit an intracellular proliferation signal when binding with IL-2. Although IL-2Rα and IL-2Rβ may also form a receptor complex with high-affinity, the receptor complex has no biological function and cannot be activated by IL-2. In different cells, different developmental stages of the same type of cells and different states of diseases, types of the IL-2 receptor have different expression degrees, thereby forming different receptor complexes. For example, LAK cell precursors express a high-level IL-2Rβγ complex, and may attack and lyse cancer cells after being activated by IL-2. Macrophages also express the IL-2Rβγ complex, and may be activated by IL-2. Monocytes express a large amount of IL-2Rγ and a small amount of IL-2Rβ, while NK cells express a large amount of IL-2Rβ and a small amount of IL-2Rγ, which form IL-2Rβγ receptors with moderate-affinity respectively to bind with the high-concentration IL-2 to form trimers, and then the monocytes or the NK cells are activated. Activated T cells express IL-2Rα, IL-2Rβ and IL-2Rγ on their surfaces, excessive IL-2Rα is beneficial for polymerizing with IL-2Rβ first, and IL-2Rαβ binds with IL-2 and then binds with IL-2Rγ to form the high-affinity receptor IL-2 complex, thus transmitting a signal, and causing a cell proliferation reaction. After the cell reaction, IL-2Rα, IL-2Rβ and IL-2Rγ are dissociated, and cells are no longer sensitive to IL-2. Human tumor cells also express the IL-2 receptor, and IL-2 may inhibit the proliferation of tumor cells after binding with receptor complexes on the tumor cells. Because different cancer cells express respective special IL-2 receptor complexes, the structure of IL-2 is improved to act on the corresponding receptors on surfaces of specific tumor surfaces only, so that only cancer cells are attacked, and the damage to normal cells is reduced.

Based on this research theory, many researchers have modified IL-2 in different directions to enhance the binding with specific receptor complexes (such as the IL-2Rβγ complex) on surfaces of tumor-related effector cells, activate cell types related to tumor killing, and minimize the binding with the high-expression IL-2Rαβγ complexes on surfaces of negative immunoregulatory T cells (such as Treg cells) at the same time, which can not only enhance the efficacy of drugs, but also reduce the side effects of drugs. Existing modifications to IL-2 comprise: designing specific IL-2 mutant proteins (for example, Aron M. L. et al., Nature, 484(7395):529-33, 2012), and changing the amino acid sequence of the binding site with IL-2α, IL-2β or IL-2γ to make the spatial structure unfavorable to the interaction with IL-2α, or enhancing the interaction with IL-2Rβ or IL-2Rγ; designing an IL-2/anti-IL-2 antibody (or IL-2 receptor) complex (for example, Jared E. L. et al., Jimunother Cancer, 8(1): e000673, 2020), and using anti-IL-2 antibody to cover the binding site with IL-2R specifically, so as to realize the change of IL-2 function and the prolongation of in-vivo half-life period; carrying out fusion expression of IL-2 with Fc or human serum albumin (HSA) to prolong the in-vivo half-life period of IL-2 (for example, Jianyong Lei et al., Protein Expression and Purification, 84(1): 154-160, 2012); combining site-directed mutation with HSA/Fc fusion to simultaneously realize the change of function and the prolongation of half-life period (for example, CN112724259A); and carrying out PEGylation of IL-2 by non-site-directed coupling to prolong the half-life period of IL-2 (for example, Deborah H. C et al., Clin Cancer Res, 22(3):680-90, 2016).

The above research on the modifications to IL-2 have the following defects.

1. Simple site-directed mutation of amino acid may weaken the binding ability with IL-2Rα, or enhance the binding ability with IL-2Rβ or IL-2Rγ, but cannot effectively prolong the half-life period of molecules. Moreover, mutant products are easy to produce an immunogenic reaction in vivo, which easily reduces the biological activity of the products, and generates greater toxicity risks.

2. Simple fusion expression (such as fusion with Fc or HSA) or IL-2 modification can prolong the half-life period of molecules, but has no obvious advantages compared with unmodified IL-2 in practical use. Fusion expression can only be realized by modification of a fusion molecule at the N-terminal or the C-terminal of the target protein, and cannot realize the optimization of the modification site.

3. Some part of IL-2 subjected to site-directed mutation combined with fusion expression does not show special advantages in practical application. (For example, Rodrigo Vazquez-Lombardi et al., Nat Commun, 8:15373, 2017).

4. Conventional PEGylated IL-2 by non-site-directed coupling does not show special advantages in practical application, and PEGylated IL-2 comprehensively weakening the binding ability with IL-2Rα achieves staged success, but the characteristics of non-site-directed coupling technology make the technology have the defects of difficult control of production process and quality, complicated molecular structure and complicated mechanism of action.

In view of the limitations of the IL-2 modifications above, some researchers have developed PEG site-directed coupled IL-2 (for example, WO 2019028419A1) by a codon expansion technology with an unnatural amino acid of Lys-azido, and the structural formula is as follows:

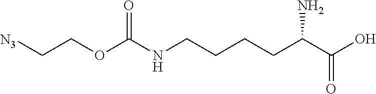 [SMILES: [N-]=[N+]=NCCOC(=O)NCCCC[C@H](N)C(=O)O]

An azide structure (—N₃) at the end of the Lys-azido can be chemically linked with a carrier drug (such as PEG) modified with an alkyne-containing structure (such as BCN, i.e.

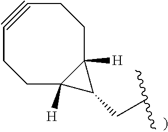 [SMILES: *C[C@H]1[C@H]2CCC#CCC[C@@H]12]

to obtain a conjugate (for example, Chinese patent CN 103153927B), which has very high specific selectivity. However, alkyne structures with high costs need to be introduced in this coupling method and chemical modification method, and an acceptable drug-antibody coupling ratio may be obtained only when an equivalent weight of use is large, which increases corresponding production costs, and has a complicated technological process and harsh technological conditions.

To sum up, there are still many shortcomings in current IL-2 modifications, and further research are needed.

### SUMMARY

Aiming at the defects in the existing modifications of IL-2, one object of the present invention is to provide a human interleukin 2-polyethylene glycol conjugate, which uses a series of brand-new unnatural amino acids to mutate one or more natural amino acids in the amino acid sequence of the recombinant human interleukin-2 in a site-directed manner, and then couples polyethylene glycol (PEG) to the unnatural amino acids in a site-directed manner through an oximation reaction, thereby forming the conjugate of the present invention.

Another object of the present invention is to provide use of the human interleukin 2-polyethylene glycol conjugate. The human interleukin 2-polyethylene glycol conjugate provided by the present invention may be used for treating diseases such as malignant solid tumors and hematologic tumors.

In a first aspect, the present invention provides a human interleukin 2-polyethylene glycol conjugate, comprising a recombinant human interleukin 2 containing at least one unnatural amino acid and PEG coupled to the at least one unnatural amino acid;
wherein, the unnatural amino acid is a compound containing a carbonyl terminal group and having a structure as shown in formula (I) or an enantiomer thereof, and the PEG is coupled to the at least one unnatural amino acid by forming an oxime bond between the carbonyl terminal group and the PEG containing a hydroxylamine terminal group (i.e., aminoxy),

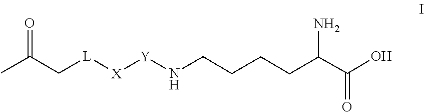

wherein X represents —O—, —S—, —NH— or —CH₂—, Y represents —O—, —S—, —C(O)—, —S(O)— or —CH₂—, and L represents substituted or unsubstituted C0-C20 linear or branched alkylene, one or more —CH₂— are optionally substituted by one or more of —O—, —S—, —NH—, —C(O)— and —S(O)—;
when X represents —O— and Y represents —C(O)—, L does not represent —CH₂CH₂—; and
when L represents a substituent, the substituent may be selected from one or more of hydroxyl, sulfhydryl, halogen, nitro, cyano, alkyl, alkenyl, alkynyl, alkoxy, acyl, acylamino, carboxyl, ester, amino, sulfonyl, sulfinyl, cycloalkyl, heterocyclyl, aryl and heteroaryl.

As shown in Example 9, the inventor of the present invention found that, besides the high cost and the complicated process, the azide structure (—N₃) at the end of Lys-azido could be easily reduced into an amino structure (—NH₂) (as shown in formula 1) when the azide structure was inserted into a recombinant human interleukin 2, thus losing coupling activity, so that the reduction reaction reduces a yield in a conjugate preparation process.

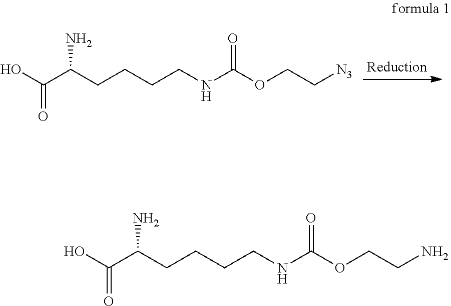 [SMILES: [N-]=[N+]=NCCOC(=O)NCCCC[C@@H](N)C(=O)O ; NCCOC(=O)NCCCC[C@@H](N)C(=O)O]

Carbonyl is introduced at the end of the unnatural amino acid according to the present invention as an active reaction group, so that the unnatural amino acid is not only novel in structure, simple and convenient to prepare, but also mild in coupling conditions and low in production cost, and is not easy to lose reaction activity due to a structural change when being inserted into a protein. In addition, the unnatural amino acid according to the present invention further contains alkylene with a certain chain length, so that the compound has better flexibility and is easier to form conjugates.

In the conjugate provided by the present invention, the unnatural amino acid contained in the recombinant human interleukin 2 contains a carbonyl terminal group, while the used PEG contains a hydroxylamine terminal group, having a structure as shown in formula (II),

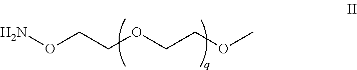

the carbonyl in the unnatural amino acid can be oximated with the hydroxylamine group in the PEG to form an oxime bond, with a structure as shown in formula (III), thus coupling the PEG to the unnatural amino acid.

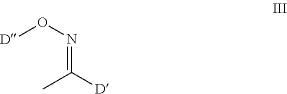

in formula (III), D′ represents a residue of the recombinant human interleukin-2 of the present invention from which the carbonyl part of the unnatural amino acid is removed, and D″ represents the PEG from which the “NH₂—O—” terminal group is removed.

Compared with the wild-type IL-2 or the commercially available recombinant human IL-2, the conjugate provided by the present invention has a reduced binding force with IL-2Rα, retains a binding activity with IL-2Rβγ, retains an ability to activate and expand CD8⁺ T cells through the activation of the CD8⁺ T cells by an IL-2Rβγ complex, inhibits the expansion of Treg cells at the same time, has a significantly prolonged half-life in vivo, and can effectively promote immunity and inhibit tumors. Moreover, the conjugate provided by the present invention has higher coupling rate and better stability.

In some preferred embodiments of the present invention, the recombinant human interleukin 2 is a protein shown in SEQ ID NO: 3 or a functional active fragment thereof.

In some preferred embodiments of the present invention, in the recombinant human interleukin 2 containing at least one unnatural amino acid, a position of the at least one unnatural amino acid is selected from one or more sites corresponding to sites P34, K35, T37, R38, L40, T41, F42, K43, F44, Y45, E61, E62, K64, P65, E67, E68, N71, L72 and Y107 of SEQ ID NO: 2. In some more preferred embodiments of the present invention, in the recombinant human interleukin 2 containing at least one unnatural amino acid, the position of the at least one unnatural amino acid is selected from one or more sites corresponding to sites K35, T41, K43, Y45, E61, K64 and P65 of SEQ ID NO: 2.

In the unnatural amino acid according to the present invention, “C0-Cn” includes C0-C1, C0-C2, . . . , C0-Cn, and C0 indicates that the group does not exist, and C atoms at two ends of the group are directly connected to form a bond. For example, the “C0-C6” group refers to that there are 0 to 6 carbon atoms in this part, which means that the group does not exist, the group contains 1 carbon atom, 2 carbon atoms, 3 carbon atoms, 4 carbon atoms, 5 carbon atoms or 6 carbon atoms. The “C6-C10” group refers to that there are 6 to 10 carbon atoms in this part, which means that the group contains 6 carbon atoms, 7 carbon atoms, 8 carbon atoms, 9 carbon atoms or 10 carbon atoms.

In some preferred embodiments of the present invention, the substituent may be selected from one or more of hydroxyl, sulfhydryl, halogen, nitro, cyano, C1-C6 alkyl, C1-C6 alkoxy, acyl, acylamino, carboxyl, ester, amino, sulfonyl, sulfinyl, C3-C8 cycloalkyl, C3-C8 heterocyclyl, C6-C20 aryl and C4-C10 heteroaryl.

In some preferred embodiments of the present invention, L may represent C0-C10 linear or branched alkylene, wherein one or more —CH₂— may be optionally substituted by —O—. In some more preferred embodiments of the present invention, L may represent C0-C6 linear alkylene, wherein one or more —CH₂— may be optionally substituted by —O—.

In some preferred embodiments of the present invention, X may represent —O—, —S—, —NH— or —CH₂—.

In some preferred embodiments of the present invention, Y may represent —C(O)—.

In some preferred embodiments of the present invention, the unnatural amino acid may be a compound with a structure as shown in formula (I-1),

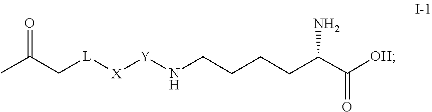

wherein X, Y and L are each independently as defined in any one of the above technical solutions.

In some preferred embodiments of the present invention, the unnatural amino acid is a compound with a structure as shown in formula (I-2), formula (I-3), formula (I-4) or formula (I-5),

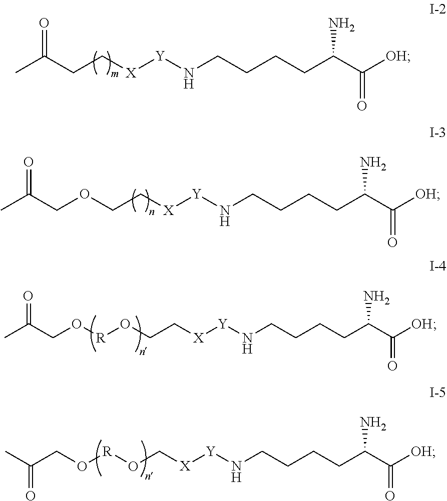

wherein X and Y are each independently as defined in any one of the above technical solutions, m and n each independently represent an integer of 0 to 8 (for example, 0, 1, 2, 3, 4, 5, 6, 7 or 8), preferably represent an integer of 0 to 5, and more preferably represent 0, 1, 2 or 3;
when X represents —O— and Y represents —C(O)—, m does not represent 1; and R represents C1-C4 linear or branched alkylene, and preferably represents —CH₂—, —CH₂CH₂-or —CH₂CH₂CH₂—; n′ represent an integer of 0 to 5 (for example, 0, 1, 2, 3, 4 or 5), preferably represent an integer of 0 to 3, and more preferably represent 0, 1 or 2.

The unnatural amino acid according to the present invention includes optically pure enantiomer and racemate.

In some more preferred embodiments of the present invention, the unnatural amino acid according to the present invention is a compound with one of the structures as follows:

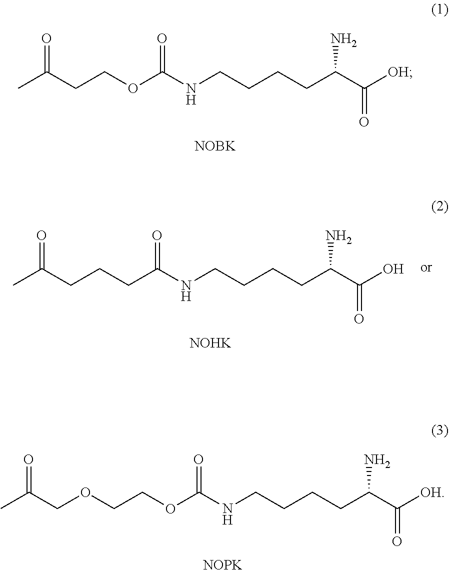

In some preferred embodiments of the present invention, the PEG containing the hydroxylamine (“NH₂—O—”) terminal group may have a molecular weight of 10 KD to 100 KD, including, but being not limited to molecular weight values of about 10 KD, 20 KD, 30 KD, 40 KD, 50 KD, 60 KD, 70 KD, 80 KD, 90 KD, 100 KD or a molecular weight interval of any combination. In some more preferred embodiments of the present invention, the molecular weight of the PEG containing the “NH₂—O—” terminal group may be 20 KD to 50 KD.

In some preferred embodiments of the present invention, the recombinant human interleukin 2 containing at least one unnatural amino acid according to the present invention may be prepared by a codon expansion technology or by means of chemical synthesis. In some more preferred embodiments of the present invention, the recombinant human interleukin 2 containing at least one unnatural amino acid according to the present invention is prepared by the codon expansion technology, wherein the codon expansion technology is implemented in Escherichia coli.

The codon expansion technology according to the present invention may specifically include the following steps: comparing the mutated nucleic acid molecule of the recombinant human interleukin 2 with the nucleic acid molecule encoding the recombinant human interleukin 2, the mutated nucleic acid molecule is different in that: the codons of the amino acid corresponding to at least one sites of P34, K35, T37, R38, L40, T41, F42, K43, F44, Y45, E61, E62, K64, P65, E67, E68, N71, L72 and Y107 of SEQ ID NO: 2 are mutated into amber codons UAG; the mutated nucleic acid molecule is expressed in Escherichia coli, and meanwhile, a lysine analogue (such as NOPK) containing the carbonyl of the present invention is incorporated into the expressed recombinant human interleukin 2 through an orthogonal tRNA synthetase/tRNA pair. The working principle of the codon expansion system is as follows: tRNA^{Pyl} cannot use lysyl tRNA enzyme of host cells, but can only be acylated by tRNA^{Pyl} RS; tRNA^{Pyl} RS can only acylate tRNA^{Pyl}, but cannot acylate other tRNAs, that is, there is orthogonality between tRNA^{Pyl} and tRNA^{Pyl} RS, and only tRNA^{Pyl} RS can acylate the corresponding unnatural amino acids to this orthogonal tRNA, and can only acylate this tRNA, but cannot acylate other tRNAs. The codon expansion system can make the lysine analogues containing the carbonyl correspond to the amber codons UAG (that is, the codons corresponding to tRNA^{Pyl} are UAG), so that the lysine analogues containing the carbonyl can be introduced into IL-2 at a specific site.

In some preferred embodiments of the present invention, after expressing the mutated recombinant human interleukin 2 in Escherichia coli, steps of protein denaturation, renaturation and ultrafiltration are also included.

In some preferred embodiments of the present invention, the oximation reaction of the unnatural amino acid in the recombinant human interleukin 2 with the PEG containing the hydroxylamine may include the following process: before the oximation reaction, adjusting the pH of the mutated recombinant human interleukin 2 solution to about 3.5 to 4.5 (for example, the pH is adjusted to about 4.0 using 2M acetic acid solution), and adjusting the protein concentration to 0.5 mg/ml to 1.5 mg/ml (for example, using 20 mM sodium acetate buffer at pH 4.0), feeding according to a certain molar ratio (for example, protein: PEG=1:15), fully dissolving, sealing, and shaking in a constant temperature shaker for reaction (for example, reaction for 30 hours to 60 hours). After the reaction, the coupling situation may be analyzed by a conventional detection method (for example, RP-HPLC).

In some preferred embodiments of the present invention, after the oximation reaction (i.e. the coupling reaction), the reaction solution contains some unreacted IL-2, impurity proteins and unreacted PEG, so the reaction solution may be further purified by cation exchange chromatography. For example, the following purification process may be adopted: chromatographic medium: Capto MMC; balance buffer: 20 mM sodium citrate buffer (pH=3.0), elution buffer: 20 mM sodium citrate buffer −1 M NaCl (pH=7.8), wherein the pH of the coupling reaction solution is adjusted to 3.0±0.2 with the balance buffer, and the conductivity is equal to or less than 5 ms/cm, then the reaction solution is loaded to Capto MMC to subject linear elution with the elution buffer (0% to 100% elution buffer, 20 CV) to collect target protein components. A target protein sample with a purity of about 95% can be obtained by this purification process.

In a second aspect, the present invention also provides use of the human interleukin 2-polyethylene glycol conjugate according to any one of the above technical solutions in preparation of a drug for promoting immunity, preventing and/or treating solid tumors (especially malignant solid tumors) and hematologic tumors, and/or expanding CD8⁺ T cells.

In some preferred embodiments of the present invention, the solid tumors refer to bladder cancer, bone cancer, brain cancer, breast cancer, colorectal cancer, esophageal cancer, ocular cancer, head and neck cancer, kidney cancer, lung cancer, melanoma, ovarian cancer, pancreatic cancer or prostate cancer.

In some preferred embodiments of the present invention, the hematologic tumors refer to chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B-cell lymphoma, nodal marginal zone B-cell lymphoma, Burkitt lymphoma, non-Burkitt high-grade B-cell lymphoma, primary mediastinal B-cell lymphoma (PMBL), immunoblastic large cell lymphoma, precursor B-cell lymphoblastic lymphoma, B-cell prolymphocytic leukemia, lymphplasmacytic lymphoma, splenic marginal zone lymphoma, plasma cell myeloma, plasmacytoma, mediastinal (thymic) large B-cell lymphoma, intravascular large B-cell lymphoma, primary effusion lymphoma, or lymphomatoid granulomatosis.

In a third aspect, the present invention also provides a kit, which includes any one of the above-mentioned human interleukin 2-polyethylene glycol conjugate.

In a fourth aspect, the present invention also provides a method for preventing and/or treating solid tumors (especially malignant solid tumors) and hematologic tumors, comprising the step of administering a therapeutically effective amount of any one of the above-mentioned human interleukin 2-polyethylene glycol conjugate to a patient in need.

In some preferred embodiments of the present invention, the solid tumors refer to bladder cancer, bone cancer, brain cancer, breast cancer, colorectal cancer, esophageal cancer, ocular cancer, head and neck cancer, kidney cancer, lung cancer, melanoma, ovarian cancer, pancreatic cancer or prostate cancer.

In some preferred embodiments of the present invention, the hematologic tumors refer to chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B-cell lymphoma, nodal marginal zone B-cell lymphoma, Burkitt lymphoma, non-Burkitt high-grade B-cell lymphoma, primary mediastinal B-cell lymphoma (PMBL), immunoblastic large cell lymphoma, precursor B-cell lymphoblastic lymphoma, B-cell prolymphocytic leukemia, lymphplasmacytic lymphoma, splenic marginal zone lymphoma, plasma cell myeloma, plasmacytoma, mediastinal (thymic) large B-cell lymphoma, intravascular large B-cell lymphoma, primary effusion lymphoma, or lymphomatoid granulomatosis.

In some preferred embodiments of the present invention, the human interleukin 2-polyethylene glycol conjugate may be administered alone or in combination with one or more other anti-tumor drugs.

The technical solutions provided by the present invention have the following advantages:

(1) The novel unnatural amino acid of the present invention can be introduced to a designated site through the codon expansion technology, so as to realize the accurate site-directed coupling of the PEG and the interleukin-2, and overcomes the defects that the traditional random coupling method cannot accurately couple, and has high product homogeneity.

(2) The unnatural amino acid of the present invention is a lysine analogue containing a terminal carbonyl in the structure thereof, compared with the common lysine analogue containing an azide group (for example, Lys-azido), the unnatural amino acid is simpler to prepare, safer, less likely to be inactivated when being inserted into the protein, higher in binding rate with the PEG, and better in stability of the obtained conjugate, and the coupling efficiency can still exceed 95% after denaturation and renaturation of the recombinant protein inclusion body.

(3) Through the design and screening of mutation sites, the present invention obtains the interleukin-2 mutation site which can reduce the binding activity of IL-2Rα and keep the binding activity of IL-2Rβ and IL-2Rγ relatively unchanged, so that the site-directed modified human interleukin 2-polyethylene glycol conjugate can specifically promote the proliferation of CD8⁺ T cells in the tumor microenvironment, but has no obvious effect on the proliferation of CD4⁺ T cells, thereby being beneficial to the immunotherapy of tumors.

(4) The conjugate of the present invention realizes the extension of the half-life of IL-2 in vivo through PEG coupling, thereby reducing the administration frequency of patients.

### DESCRIPTION OF THE DRAWINGS

FIG. 1 is a schematic diagram of an expression plasmid NB1S3-WT.

FIG. 2 is a schematic diagram of a helper plasmid NB1W.

FIG. 3 is an SDS-PAGE electrophoretogram of fermentation products obtained by rhIL-2 expression strains with different mutation sites obtained in Example 2 after adding an unnatural amino acid NOPK (the arrows indicate bands position of target products); wherein Lane 1: rhIL2-K35-BL21 strains fed with NOPK are crushed and precipitated after centrifugation; Lane 2: rhIL2-T41-BL21 strains fed with NOPK are crushed and precipitated after centrifugation; Lane 3: rhIL2-K43-BL21 strains fed with NOPK are crushed and precipitated after centrifugation; Lane 4: rhIL2-Y45-BL21 strains fed with NOPK are crushed and precipitated after centrifugation; Lane 5: rhIL2-E61-BL21 strains fed with NOPK are crushed and precipitated after centrifugation; Lane 6: rhIL2-K64-BL21 strains fed with NOPK are crushed and precipitated after centrifugation; Lane 7: rhIL2-P65-BL21 strains fed with NOPK are crushed and precipitated after centrifugation; and Lane 8: rhIL2-Y45-BL21 strains fed with NOPK are precipitated and washed to obtain inclusion bodies.

FIG. 4A and FIG. 4B are RP-HPLC profiles of mutant rhIL-2 before and after coupling with PEG in Example 3 respectively; wherein, FIG. 4A shows each mutant rhIL-2 before coupling, wherein the main peak of the target protein is at about 22.5 min; and FIG. 4B shows each mutant rhIL-2 (PEG is 30 KD PEG) after coupling, wherein the main peak of the target protein is at about 21 min.

FIG. 5 is an RP-HPLC profile of the conjugate 30 KD PEG-rhIL2-Y45 in Example 4 before and after column chromatography.

FIG. 6 is a mass spectrogram of rhGH-V91 in Example 9.

### EMBODIMENT

The technical solutions of the present invention will be further described in detail hereinafter with reference to the specific examples.

Unless otherwise specified, the reagents or raw materials used in the preparation examples and examples of the present invention are all commercially available products; and the experimental methods used are all conventional methods in this field unless otherwise specified.

Human YT cells are disclosed in the literature “Yodoi, J. et al., (1985). TCGF (IL 2)-receptor inducing factor(s). I. Regulation of IL 2 receptor on a natural killer-like cell line (YT cells). Journal of Immunology, 134(3), 1623-1630”, and can be obtained by the public from Novocodex Biopharmaceuticals Co., Ltd.

### Preparation Example 1 Preparation of Unnatural Amino Acid NOBK

The structural formula of NOBK was as follows:

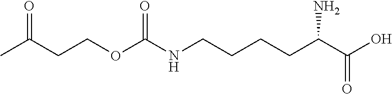 [SMILES: CC(=O)CCOC(=O)NCCCC[C@H](N)C(=O)O]

The reaction process was shown in the following figure:

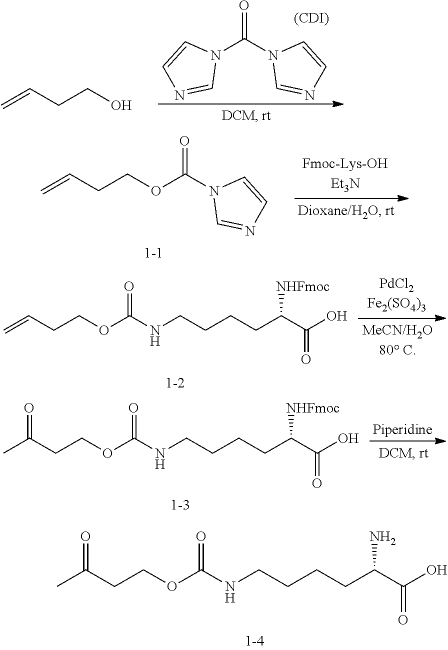

a) 3-butene-1-ol (2.00 g, 27.74 mmol) and a solvent DCM (15.0 mL) were added into a reaction flask, and then CDI (4.50 g, 27.74 mmol) was added at 0° C. The mixture was reacted at room temperature for 18 hours, and then added with 10 mL of water, and extracted with DCM for 3 times. Organic phases were combined, dried with anhydrous sodium sulfate, filtered, and concentrated under a reduced pressure to obtain a product 1-1 (4.50 g, yield 98%).
b) The product 1-1 (0.55 g, 3.31 mmol) and Fmoc-lysine hydrochloride (1.12 g, 2.76 mmol) were added into a reaction flask, added with 9 mL of dioxane and 3 mL of water (v/v=3:1), and then added with triethylamine (0.70 g, 6.9 mmol). The mixture was reacted at room temperature for 24 hours, added with 20 mL of 1 M HCl solution to adjust the pH value to be about 2, and extracted with ethyl acetate for 3 times. Organic phases were combined, dried with anhydrous sodium sulfate, filtered, concentrated under a reduced pressure, and purified by column chromatography (eluent:DCM:MeOH=30:1) to obtain a product 1-2 (0.17 g, yield 14%).
c) The product 1-2 (0.13 g, 0.28 mmol), ferric sulfate (0.17 g, 0.42 mmol) and palladium chloride (15 mg, 0.03 mmol) were added into a two-neck reaction flask, added with 3 mL of acetonitrile and 0.5 mL of water. After the mixture was refluxed at 60° C. for 24 hours, the mixture was cooled to room temperature, and extracted with DCM for 3 times. Organic phases were combined, concentrated under a reduced pressure, and purified by column chromatography (eluent:DCM:MeOH=20:1) to obtain a product 1-3 (91.0 mg, yield 68%).
d) The product 1-3 (91.0 mg, 0.19 mmol) was dissolved in DCM (2 mL) in a reaction flask, added with piperidine (36.4 mg, 0.43 mmol), reacted at room temperature for 3 hours, concentrated under a reduced pressure, and purified by column chromatography (eluent:MeOH:H₂O=40:10:1) to obtain the target product NOBK 1-4 (20.1 mg, yield 41%).

¹H NMR (400 MHz, heavy water) δ 4.20 (t, J=6.8 Hz, 2H), 3.64 (t, J=6.0 Hz, 1H), 3.02 (t, J=6.0 Hz, 2H), 2.16 (s, 3H), 2.07-1.80 (m, 4H), 1.69-1.52 (m, 2H), 1.52-1.36 (m, 2H).

### Preparation Example 2 Preparation of Unnatural Amino Acid NOHK

The structural formula of NOHK was as follows:

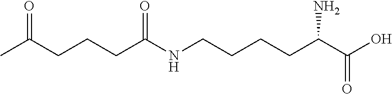 [SMILES: CC(=O)CCCC(=O)NCCCC[C@H](N)C(=O)O]

The reaction process was shown in the following figure:

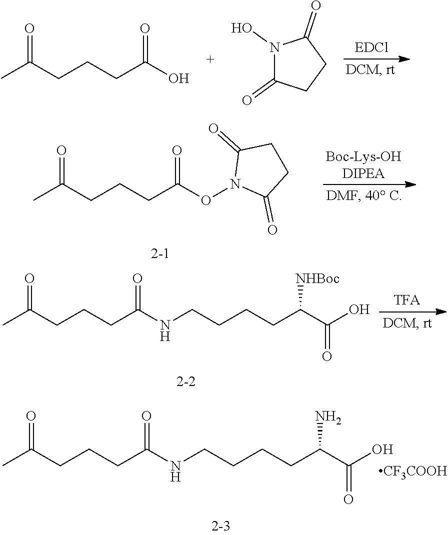

The preparation process included the following steps.
a) 5-oxohexanoic acid (2.60 g, 20.0 mmol), N-hydroxysuccinimide (2.30 g, 20.0 mmol) and EDCI (3.83 g, 20.0 mmol) were added into a reaction flask, and then a solvent DCM (100 mL) was added. The mixture was reacted at room temperature for 18 hours, washed with water for 3 times, dried with anhydrous sodium sulfate, and concentrated under a reduced pressure to obtain a product 2-1 (4.62 g, yield 102%), which was directly used in next step without purification.
b) The product 2-1 (4.62 g, 20.35 mmol) and Boc-lysine (4.42 g, 16.96 mmol) were added into a reaction flask, and then added with DIPEA (4.07 mL, 42.4 mmol) and a solvent DMF (50 mL). The mixture was reacted at 40° C. for 24 hours, washed with water for 5 times, dried with anhydrous sodium sulfate, filtered, concentrated under a reduced pressure, and purified by column chromatography (eluent:DCM:MeOH=30:1) to obtain a product 2-2 (4.36 g, yield 60%).
c) The product 2-2 (4.36 g), a solvent DCM (10 mL) and an equivalent amount (10 mL) of trifluoroacetic acid (TFA) were added into a reaction flask. The mixture was stirred at room temperature for 20 minutes, concentrated under a reduced pressure, then added with an acetonitrile solvent for multiple times to remove excess trifluoroacetic acid under reduced pressure concentration to obtain the target product NOHK 2-3 (4.10 g, yield 91%).

¹H NMR (400 MHz, heavy water) δ 3.75 (t, J=6.0 Hz, 1H), 3.21 (t, J=6.8 Hz, 2H), 2.61 (t, J=7.2 Hz, 2H), 2.26 (d, J=7.4 Hz, 2H), 2.23 (s, 3H), 1.96-1.79 (m, 4H), 1.63-1.52 (m, 2H), 1.49-1.34 (m, 2H).

### Preparation Example 3 Preparation of Unnatural Amino Acid NOPK

The structural formula of NOPK was as follows:

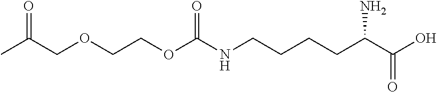 [SMILES: CC(=O)COCCOC(=O)NCCCC[C@H](N)C(=O)O]

The reaction process was shown in the following figure:

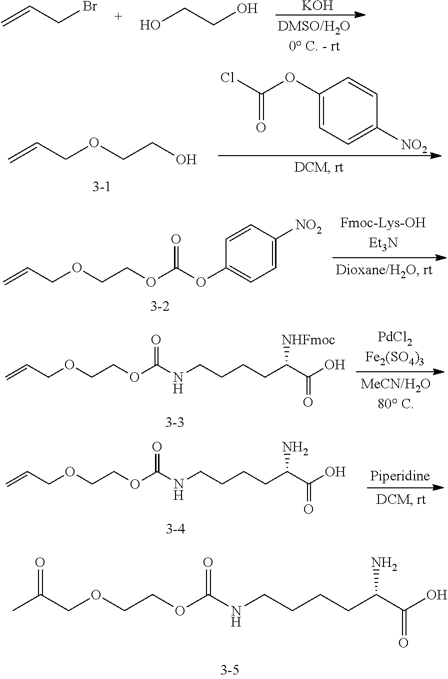

The preparation process included the following steps.

a) Bromopropylene (18.15 g, 0.15 mol) and DMSO (60 mL) were added into a reaction flask, cooled to 0° C., added with glycol (37.26 g, 0.6 mol), DMSO (120 mL), KOH (10.11 g, 0.18 mol) and water (30 mL). The mixture was reacted for 1 hour at the temperature, and then heated to room temperature to react and stir for 18 hours. The reaction solution was added with water (30 mL), and extracted with DCM for 3 times. Organic phases were combined, washed with water for 2 times, dried with anhydrous sodium sulfate, filtered, concentrated under a reduced pressure at about 0° C., and finally purified by column chromatography (eluent:PE:PA=10:1) to obtain a product 3-1 (6.30 g, yield 25%).

b) P-nitrophenyl chloroformate (1.0 g, 4.90 mmol) and a solvent DCM (20.0 mL) were added into a reaction flask, cooled to 0° C., added with the product 3-1 (0.50 g, 4.90 mmol) and pyridine (0.41 g, 5.14 mmol). After the mixture was heated to room temperature to react and stir for 18 hours, the reaction solution was added with saturated sodium carbonate solution (10 mL), and extracted with DCM (50 mL) for 3 times. Organic phases were combined, washed with water for 2 times, dried with anhydrous sodium sulfate, filtered, concentrated under a reduced pressure, and purified by column chromatography (eluent:PE:PA=2:1) to obtain a product 3-2 (1.00 g, yield 76%).

c) The product 3-2 (1.00 g, 3.74 mmol) and Fmoc-lysine hydrochloride (1.38 g, 3.40 mmol) were added into a reaction flask, added with a solvent dioxane (15 mL) and water (5 mL), and then added with triethylamine (0.86 g, 8.50 mmol). The mixture was reacted at room temperature for 24 hours, added with an with an appropriate amount of 1 M HCl solution to adjust the pH value to be about 2, and extracted with DCM, concentrated under a reduced pressure, and purified by column chromatography (eluent:DCM:MeOH=30:1) to obtain a product 3-3 (1.01 g, yield 54%).

d) The product 3-3 (6.80 g, 13.69 mmol), ferric sulfate (8.21 g, 20.54 mmol) and palladium chloride (0.48 g, 2.74 mmol) were added into a two-neck reaction flask, added with a solvent acetonitrile (60 mL) and water (10 mL). After the mixture was refluxed at 80° C. for 48 hours, the mixture was concentrated under a reduced pressure, extracted with DCM for 3 times, and purified by column chromatography (eluent:DCM:MeOH=20:1) to obtain a product 3-4 (2.10 g, yield 30%).

e) The product 3-4 (8.00 g, 15.61 mmol) was dissolved in DCM (150 mL) in a reaction flask, added with piperidine (4.00 g, 46.82 mmol), reacted at room temperature for 3 hours, concentrated under a reduced pressure, and purified by column chromatography (eluent:MeOH:H₂O=40:10:1) to obtain the target product NOPK 3-5 (2.80 g, yield 62%).

¹H-NMR (400 MHz, heavy water) b 4.41-4.25 (m, 2H), 4.23-4.10 (m, 2H), 3.85-3.66 (m, 2H), 3.66-3.60 (m, 1H), 3.06 (t, J=8.4 Hz, 2H), 2.07 (s, 3H), 1.85-1.70 (m, 2H), 1.54-1.41 (m, 2H), 1.38-1.24 (m, 2H).

### Example 1 Construction of Expression Strain Expressing Recombinant Human IL-2 (rhIL-2) Inserted with Unnatural Amino Acid at Directed Site

### 1. Acquisition of Expression Plasmid NB1S3-WT of Wild-Type Recombinant Human IL-2

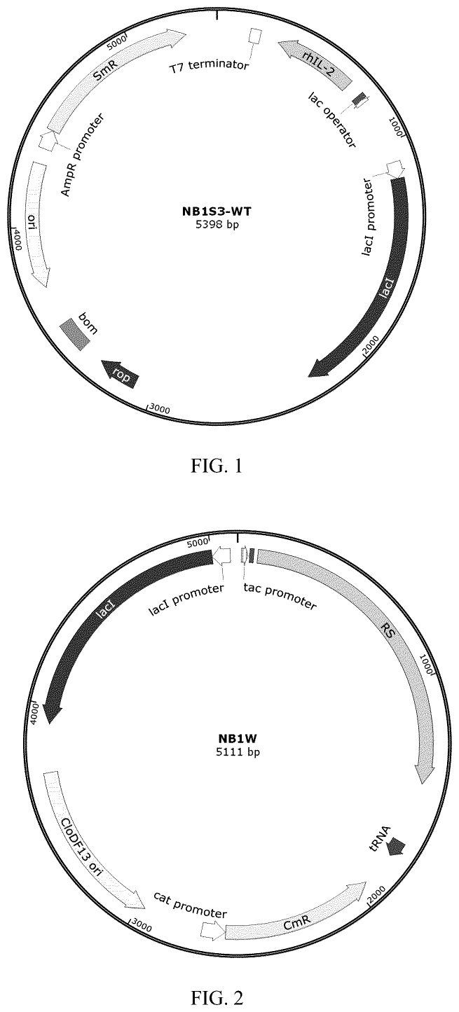

The precursor protein sequence (GenBank ID: CAA25292.1) of Homo sapiens IL-2 was obtained from the National Center for Biotechnology Information (NCBI), as shown in SEQ ID NO: 1. The N-terminal of the precursor sequence contained a signal peptide sequence consisting of 20 amino acids, which could be excised in the processing and maturing process of IL-2 protein molecules, so that a protein sequence (SEQ ID NO: 2) of mature Homo sapiens IL-2 was obtained after the signal peptide sequence was removed. According to the literature report (Liang S. M. et al. Journal of Biological Chemistry, 261(1): 334-337, 1986), the protein sequence of mature Homo sapiens IL-2 contained three Cysteines (Cys), wherein two Cysteines at the 58^{th} site and the 105^{th} site could form disulfide bonds, which were very important for the biological activity of Homo sapiens IL-2. The Cys at the 125^{th} site did not participate in the formation of disulfide bonds, but could interfere with the formation of normal disulfide bonds in the renaturation process of the protein inclusion body of recombinant Homo sapiens IL-2, so that the Cys at the 125^{th} site could be mutated into a Serine (Ser), and the renaturation efficiency was improved without significantly affecting the activity. Meanwhile, in order to express a recombinant protein in Escherichia coli, it was necessary to add methionine (Met) to the N-terminal of the protein sequence for initiating the translation of the protein, so that a protein sequence (SEQ ID NO: 3) of mature recombinant human IL-2 was obtained. A gene sequence (SEQ ID NO: 4) encoding the recombinant human IL-2 was obtained by the reverse translation process of amino acids and codons and the optimization of the codons, and an encoding gene of the recombinant human IL-2 was obtained by whole gene synthesis. Then, the gene was linked to a NB1 S3 expression vector by one-step sub-cloning (the expression vector was modified from a commercial vector pET-21a, and the screening marker of the ampicillin resistance gene was replaced by a spectinomycin resistance gene amplified from a commercial vector pCDF-duet1 by PCR), and an expression plasmid NB1 S3-WT of wild-type recombinant human IL-2 was obtained (referring to FIG. 1), with a sequence shown in SEQ ID NO: 5.

SEQ ID NO: 1
MYRMQLLSCIALSLALVTNSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT
SEQ ID NO: 2
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT
SEQ ID NO: 3
MAPTSSSTKKTQLQLEHLLLDLQMILNGINNYKNPKLTRMLTFKF
YMPKKATELKHLQCLEEELKPLEEVLNLAQSKNFHLRPRDLISNI
NVIVLELKGSETTFMCEYADETATIVEFLNRWITFSQSIISTLT
SEQ ID NO: 4
ATGGCGCCTACATCCAGCTCGACCAAAAAGACGCAGCTGCAACTG
GAACACCTGCTCCTGGATCTGCAAATGATTCTTAACGGTATCAAT
AACTACAAAAATCCGAAACTGACCCGTATGCTGACGTTTAAATTC
TATATGCCAAAGAAAGCGACCGAGCTGAAACATCTGCAGTGCCTG
GAAGAGGAACTGAAACCGCTGGAGGAAGTTTTGAACCTGGCTCAG
TCTAAAAACTTTCACCTGCGCCCTCGTGACCTGATTTCCAATATC
AACGTGATTGTTCTGGAACTGAAAGGCTCTGAAACCACGTTTATG
TGCGAGTACGCCGATGAAACCGCCACGATTGTGGAATTTCTGAAT
CGCTGGATCACCTTCTCCCAGAGCATTATTAGCACGCTGACCTAA
SEQ ID NO: 5
GGCGAGAAAGGAAGGGAAGAAAGCGAAAGGAGCGGGCGCTAGGGC
GCTGGCAAGTGTAGCGGTCACGCTGCGCGTAACCACCACACCCGC
CGCGCTTAATGCGCCGCTACAGGGCGCGTCCCATTCGCCAATCCG
GATATAGTTCCTCCTTTCAGCAAAAAACCCCTCAAGACCCGTTTA
GAGGCCCCAAGGGGTTATGCTAGTTATTGCTCAGCGGTGGCAGCA
GCCAACTCAGCTTCCTTTCGGGCTTTGTTAGCAGCCGGATCTCAG
TGGTGGTGGTGGTGGTGCTCGAGTTAGGTCAGCGTGCTAATAATG
CTCTGGGAGAAGGTGATCCAGCGATTCAGAAATTCCACAATCGTG
GCGGTTTCATCGGCGTACTCGCACATAAACGTGGTTTCAGAGCCT
TTCAGTTCCAGAACAATCACGTTGATATTGGAAATCAGGTCACGA
GGGCGCAGGTGAAAGTTTTTAGACTGAGCCAGGTTCAAAACTTCC
TCCAGCGGTTTCAGTTCCTCTTCCAGGCACTGCAGATGTTTCAGC
TCGGTCGCTTTCTTTGGCATATAGAATTTAAACGTCAGCATACGG
GTCAGTTTCGGATTTTTGTAGTTATTGATACCGTTAAGAATCATT
TGCAGATCCAGGAGCAGGTGTTCCAGTTGCAGCTGCGTCTTTTTG
GTCGAGCTGGATGTAGGCGCCATATGTATATCTCCTTCTTAAAGT
TAAACAAAATTATTTCTAGAGGGGAATTGTTATCCGCTCACAATT
CCCCTATAGTGAGTCGTATTAATTTCGCGGGATCGAGATCTCGAT
CCTCTACGCCGGACGCATCGTGGCCGGCATCACCGGCGCCACAGG
TGCGGTTGCTGGCGCCTATATCGCCGACATCACCGATGGGGAAGA
TCGGGCTCGCCACTTCGGGCTCATGAGCGCTTGTTTCGGCGTGGG
TATGGTGGCAGGCCCCGTGGCCGGGGGACTGTTGGGCGCCATCTC
CTTGCATGCACCATTCCTTGCGGCGGCGGTGCTCAACGGCCTCAA
CCTACTACTGGGCTGCTTCCTAATGCAGGAGTCGCATAAGGGAGA
GCGTCGAGATCCCGGACACCATCGAATGGCGCAAAACCTTTCGCG
GTATGGCATGATAGCGCCCGGAAGAGAGTCAATTCAGGGTGGTGA
ATGTGAAACCAGTAACGTTATACGATGTCGCAGAGTATGCCGGTG
TCTCTTATCAGACCGTTTCCCGCGTGGTGAACCAGGCCAGCCACG
TTTCTGCGAAACGCGGGAAAAAGTGGAAGCGGCGATGGCGGAGCT
GAATTACATTCCCAACCGCGTGGCACAACAACTGGGGGGCAAACA
GTCGTTGCTGATTGGCGTTGCCACCTCCAGTCTGGCCCTGCACGC
GCCGTCGCAAATTGTCGCGGCGATTAAATCTCGCGCCGATCAACT
GGGTGCCAGCGTGGTGGTGTCGATGGTAGAACGAAGCGGCGTCGA
AGCCTGTAAAGCGGCGGTGCACAATCTTCTCGCGCAACGCGTCAG
TGGGCTGATCATTAACTATCCGCTGGATGACCAGGATGCCATTGC
TGTGGAAGCTGCCTGCACTAATGTTCCGGCGTTATTTCTTGATGT
CTCTGACCAGACACCCATCAACAGTATTATTTTCTCCCATGAAGA
CGGTACGCGACTGGGCGTGGAGCATCTGGTCGCATTGGGTCACCA
GCAAATCGCGCTGTTAGCGGGCCCATTAAGTTCTGTCTCGGCGCG
TCTGCGTCTGGCTGGCTGGCATAAATATCTCACTCGCAATCAAAT
TCAGCCGATAGCGGAACGGGAAGGCGACTGGAGTGCCATGTCCGG
TTTTCAACAAACCATGCAAATGCTGAATGAGGGCATCGTTCCCAC
TGCGATGCTGGTTGCCAACGATCAGATGGCGCTGGGCGCAATGCG
CGCCATTACCGAGTCCGGGCTGCGCGTTGGTGCGGATATCTCGGT
AGTGGGATACGACGATACCGAAGACAGCTCATGTTATATCCCGCC
GTTAACCACCATCAAACAGGATTTTCGCCTGCTGGGGCAAACCAG
CGTGGACCGCTTGCTGCAACTCTCTCAGGGCCAGGCGGTGAAGGG
CAATCAGCTGTTGCCCGTCTCACTGGTGAAAAGAAAAACCACCCT
GGCGCCCAATACGCAAACCGCCTCTCCCCGCGCGTTGGCCGATTC
ATTAATGCAGCTGGCACGACAGGTTTCCCGACTGGAAAGCGGGCA
GTGAGCGCAACGCAATTAATGTAAGTTAGCTCACTCATTAGGCAC
CGGGATCTCGACCGATGCCCTTGAGAGCCTTCAACCCAGTCAGCT
CCTTCCGGTGGGCGCGGGGCATGACTATCGTCGCCGCACTTATGA
AGCTTCTATCGTCGCCGCACTTATGACTGTCTTCTTTATCATGCA
ACTCGTAGGACAGGTGCCGGCAGCGCTCTGGGTCATTTTCGGCGA
GGACCGCTTTCGCTGGAGCGCGACGATGATCGGCCTGTCGCTTGC
GGTATTCGGAATCTTGCACGCCCTCGCTCAAGCCTTCGTCACTGG
TCCCGCCACCAAACGTTTCGGCGAGAAGCAGGCCATTATCGCCGG
CATGGCGGCCCCACGGGTGCGCATGATCGTGCTCCTGTCGTTGAG
GACCCGGCTAGGCTGGCGGGGTTGCCTTACTGGTTAGCAGAATGA
ATCACCGATACGCGAGCGAACGTGAAGCGACTGCTGCTGCAAAAC
GTCTGCGACCTGAGCAACAACATGAATGGTCTTCGGTTTCCGTGT
TTCGTAAAGTCTGGAAACGCGGAAGTCAGCGCCCTGCACCATTAT
GTTCCGGATCTGCATCGCAGGATGCTGCTGGCTACCCTGTGGAAC
ACCTACATCTGTATTAACGAAGCGCTGGCATTGACCCTGAGTGAT
TTTTCTCTGGTCCCGCCGCATCCATACCGCCAGTTGTTTACCCTC
ACAACGTTCCAGTAACCGGGCATGTTCATCATCAGTAACCCGTAT
CGTGAGCATCCTCTCTCGTTTCATCGGTATCATTACCCCCATGAA
CAGAAATCCCCCTTACACGGAGGCATCAGTGACCAAACAGGAAAA
AACCGCCCTTAACATGGCCCGCTTTATCAGAAGCCAGACATTAAC
GCTTCTGGAGAAACTCAACGAGCTGGACGCGGATGAACAGGCAGA
CATCTGTGAATCGCTTCACGACCACGCTGATGAGCTTTACCGCAG
CTGCCTCGCGCGTTTCGGTGATGACGGTGAAAACCTCTGACACAT
GCAGCTCCCGGAGACGGTCACAGCTTGTCTGTAAGCGGATGCCGG
GAGCAGACAAGCCCGTCAGGGCGCGTCAGCGGGTGTTGGCGGGTG
TCGGGGCGCAGCCATGACCCAGTCACGTAGCGATAGCGGAGTGTA
TACTGGCTTAACTATGCGGCATCAGAGCAGATTGTACTGAGAGTG
CACCATATATGCGGTGTGAAATACCGCACAGATGCGTAAGGAGAA
AATACCGCATCAGGCGCTCTTCCGCTTCCTCGCTCACTGACTCGC
TGCGCTCGGTCGTTCGGCTGCGGCGAGCGGTATCAGCTCACTCAA
AGGCGGTAATACGGTTATCCACAGAATCAGGGGATAACGCAGGAA
AGAACATGTGAGCAAAAGGCCAGCAAAAGGCCAGGAACCGTAAAA
AGGCCGCGTTGCTGGCGTTTTTCCATAGGCTCCGCCCCCCTGACG
AGCATCACAAAAATCGACGCTCAAGTCAGAGGTGGCGAAACCCGA
CAGGACTATAAAGATACCAGGCGTTTCCCCCTGGAAGCTCCCTCG
TGCGCTCTCCTGTTCCGACCCTGCCGCTTACCGGATACCTGTCCG
CCTTTCTCCCTTCGGGAAGCGTGGCGCTTTCTCATAGCTCACGCT
GTAGGTATCTCAGTTCGGTGTAGGTCGTTCGCTCCAAGCTGGGCT
GTGTGCACGAACCCCCCGTTCAGCCCGACCGCTGCGCCTTATCCG
GTAACTATCGTCTTGAGTCCAACCCGGTAAGACACGACTTATCGC
CACTGGCAGCAGCCACTGGTAACAGGATTAGCAGAGCGAGGTATG
TAGGCGGTGCTACAGAGTTCTTGAAGTGGTGGCCTAACTACGGCT
ACACTAGAAGGACAGTATTTGGTATCTGCGCTCTGCTGAAGCCAG
TTACCTTCGGAAAAAGAGTTGGTAGCTCTTGATCCGGCAAACAAA
CCACCGCTGGTAGCGGTGGTTTTTTTGTTTGCAAGCAGCAGATTA
CGCGCAGAAAAAAAGGATCTCAAGAAGATCCTTTGATCTTTTCTA
CGGGGTCTGACGCTCAGTGGAACGAAAACTCACGTTAAGGGACTG
CAGCTATTTGTTTATTTTTCTAAATACATTCAAATATGTATCCGC
TCATGAGACAATAACCCTGATAAATGCTTCAATAATATTGAAAAA
GGAAGAGTATGAGGGAAGCGGTGATCGCCGAAGTATCGACTCAAC
TATCAGAGGTAGTTGGCGTCATCGAGCGCCATCTCGAACCGACGT
TGCTGGCCGTACATTTGTACGGCTCCGCAGTGGATGGCGGCCTGA
AGCCACACAGTGATATTGATTTGCTGGTTACGGTGACCGTAAGGC
TTGATGAAACAACGCGGCGAGCTTTGATCAACGACCTTTTGGAAA
CTTCGGCTTCCCCTGGAGAGAGCGAGATTCTCCGCGCTGTAGAAG
TCACCATTGTTGTGCACGACGACATCATTCCGTGGCGTTATCCAG
CTAAGCGCGAACTGCAATTTGGAGAATGGCAGCGCAATGACATTC
TTGCAGGTATCTTCGAGCCAGCCACGATCGACATTGATCTGGCTA
TCTTGCTGACAAAAGCAAGAGAACATAGCGTTGCCTTGGTAGGTC
CAGCGGCGGAGGAACTCTTTGATCCGGTTCCTGAACAGGATCTAT
TTGAGGCGCTAAATGAAACCTTAACGCTATGGAACTCGCCGCCCG
ACTGGGCTGGCGATGAGCGAAATGTAGTGCTTACGTTGTCCCGCA
TTTGGTACAGCGCAGTAACCGGCAAAATCGCGCCGAAGGATGTCG
CTGCCGACTGGGCAATGGAGCGCCTGCCGGCCCAGTATCAGCCCG
TCATACTTGAAGCTAGACAGGCTTATCTTGGACAAGAAGAAGATC
GCTTGGCCTCGCGCGCAGATCAGTTGGAAGAATTTGTCCACTACG
TGAAAGGCGAGATCACCAAGGTAGTCGGCAAATAATGTCTAACAA
TTCGTTCAAGCCGAGGGGCCGCAAGATCCGGCCACGATGACCCGG
TCGTCGGTTCAGGGCAGGGTCGTTAAATAGCCGCTTATGTCTATT
GCTGGTTTACCGGTTTATTGACTACCGGAAGCAGTGTGACCGT

### 2. Selection of Site-Directed Mutation Site

The P34 site, the K35 site, the T37 site, the R38 site, the L40 site, the T41 site, the F42 site, the K43 site, the F44 site, the Y45 site, the E61 site, the E62 site, the K64 site, the P65 site, the E67 site, the E68 site, the N71 site, the L72 site and the Y107 site of SEQ ID NO: 2 were selected as specific sites for site mutation, and this mutant IL-2 was used as a raw material and modified at a directed site.

### 3. Design of Primer Subjected to Site-Directed Mutation and Construction of Mutant Vector

For the P34 site, the K35 site, the T37 site, the R38 site, the L40 site, the T41 site, the F42 site, the K43 site, the F44 site, the Y45 site, the E61 site, the E62 site, the K64 site, the P65 site, the E67 site, the E68 site, the N71 site, the L72 site and the Y107 site of SEQ ID NO: 2, primers capable of mutating the codons encoding the amino acid into amber codons were respectively designed, and specific primers were shown in Table 1.

TABLE 1
List of mutant primers

| Mutation | | Sequence number of |
|---|---|---|
| site and | Mutation site sequence | corresponding |
| name | (5′ → 3′ direction) | sequence listing |
| P34-F | CAATAACTACAAAAATTAGAAACTGACCCGTATGC | SEQ ID NO: 6 |
| P34-R | GCATACGGGTCAGTTTCTAATTTTTGTAGTTATTG | SEQ ID NO: 7 |
| K35-F | TAACTACAAAAATCCGTAGCTGACCCGTATGCTGA | SEQ ID NO: 8 |
| K35-R | TCAGCATACGGGTCAGCTACGGATTTTTGTAGTTA | SEQ ID NO: 9 |
| T37-F | CAAAAATCCGAAACTGTAGCGTATGCTGACGTTTA | SEQ ID NO: 10 |
| T37-R | TAAACGTCAGCATACGCTACAGTTTCGGATTTTTG | SEQ ID NO: 11 |
| R38-F | AAATCCGAAACTGACCTAGATGCTGACGTTTAAAT | SEQ ID NO: 12 |
| R38-R | ATTTAAACGTCAGCATCTAGGTCAGTTTCGGATTT | SEQ ID NO: 13 |
| L40-F | GAAACTGACCCGTATGTAGACGTTTAAATTCTATA | SEQ ID NO: 14 |
| L40-R | TATAGAATTTAAACGTCTACATACGGGTCAGTTTC | SEQ ID NO: 15 |
| T41-F | ACTGACCCGTATGCTGTAGTTTAAATTCTATATGC | SEQ ID NO: 16 |
| T41-R | GCATATAGAATTTAAACTACAGCATACGGGTCAGT | SEQ ID NO: 17 |
| F42-F | GACCCGTATGCTGACGTAGAAATTCTATATGCCAA | SEQ ID NO: 18 |
| F42-R | TTGGCATATAGAATTTCTACGTCAGCATACGGGTC | SEQ ID NO: 19 |
| K43-F | CCGTATGCTGACGTTTTAGTTCTATATGCCAAAGA | SEQ ID NO: 20 |
| K43-R | TCTTTGGCATATAGAACTAAAACGTCAGCATACGG | SEQ ID NO: 21 |
| F44-F | TATGCTGACGTTTAAATAGTATATGCCAAAGAAAG | SEQ ID NO: 22 |
| F44-R | CTTTCTTTGGCATATACTATTTAAACGTCAGCATA | SEQ ID NO: 23 |
| Y45-F | GCTGACGTTTAAATTCTAGATGCCAAAGAAAGCGA | SEQ ID NO: 24 |
| Y45-R | TCGCTTTCTTTGGCATCTAGAATTTAAACGTCAGC | SEQ ID NO: 25 |
| E61-F | TCTGCAGTGCCTGGAATAGGAACTGAAACCGCTGG | SEQ ID NO: 26 |
| E61-R | CCAGCGGTTTCAGTTCCTATTCCAGGCACTGCAGA | SEQ ID NO: 27 |
| E62-F | GCAGTGCCTGGAAGAGTAGCTGAAACCGCTGGAGG | SEQ ID NO: 28 |
| E62-R | CCTCCAGCGGTTTCAGCTACTCTTCCAGGCACTGC | SEQ ID NO: 29 |
| K64-F | CCTGGAAGAGGAACTGTAGCCGCTGGAGGAAGTTT | SEQ ID NO: 30 |
| K64-R | AAACTTCCTCCAGCGGCTACAGTTCCTCTTCCAGG | SEQ ID NO: 31 |
| P65-F | GGAAGAGGAACTGAAATAGCTGGAGGAAGTTTTGA | SEQ ID NO: 32 |
| P65-R | TCAAAACTTCCTCCAGCTATTTCAGTTCCTCTTCC | SEQ ID NO: 33 |
| E67-F | GGAACTGAAACCGCTGTAGGAAGTTTTGAACCTGG | SEQ ID NO: 34 |
| E67-R | CCAGGTTCAAAACTTCCTACAGCGGTTTCAGTTCC | SEQ ID NO: 35 |
| E68-F | ACTGAAACCGCTGGAGTAGGTTTTGAACCTGGCTC | SEQ ID NO: 36 |
| E68-R | GAGCCAGGTTCAAAACCTACTCCAGCGGTTTCAGT | SEQ ID NO: 37 |
| N71-F | GCTGGAGGAAGTTTTGTAGCTGGCTCAGTCTAAAA | SEQ ID NO: 38 |
| N71-R | TTTTAGACTGAGCCAGCTACAAAACTTCCTCCAGC | SEQ ID NO: 39 |
| L72-F | GGAGGAAGTTTTGAACTAGGCTCAGTCTAAAAACT | SEQ ID NO: 40 |
| L72-R | AGTTTTTAGACTGAGCCTAGTTCAAAACTTCCTCC | SEQ ID NO: 41 |
| Y107-F | CACGTTTATGTGCGAGTAGGCCGATGAAACCGCCA | SEQ ID NO: 42 |
| Y107-R | TGGCGGTTTCATCGGCCTACTCGCACATAAACGTG | SEQ ID NO: 43 |
| XbaI-F | TGAGCGGATAACAATTCCCCTCTAGAAATAATTTT | SEQ ID NO: 44 |
| | GTTTAAC | |
| XhoI-R | GATCTCAGTGGTGGTGGTGGTGGTGCTCGAG | SEQ ID NO: 45 |

The plasmid NB1S3-WT was subjected to double digestion with restriction endonucleases Xbal and Xhol to obtain a linearized DNA plasmid, which was used as a template. By using a high-fidelity DNA polymerase (purchased from Takara, catalog number R045A), the primer Xbal-F in Table 1 was paired with primers R of various sites, and the primer Xhol-R in Table 1 was paired with primers F of various sites. Mutant genes with amino acid codons at the K35 site, the T41 site, the K43 site, the Y45 site, the E61 site, the K64 site and the P65 site of IL-2 mutated into amber termination codons were obtained by PCR amplification and overlap PCR (for example, the linearized plasmid NB1S3-WT was used as a template, and Xbal-F and T41-R were used as a primer pair for PCR amplification to obtain an upstream fragment subjected to mutation at the T41 site; the linearized plasmid NB1S3-WT was used as a template, and Xhol-R and T41-F were used as a primer pair for PCR amplification to obtain a downstream fragment subjected to mutation at the T41 site; and then, the upstream fragment subjected to mutation at the T41 site and the downstream fragment subjected to mutation at the T41 site obtained above were used as templates, and Xbal-F and Xhol-R were used as a primer pair for overlap PCR amplification to obtain a full-length gene subjected to mutation at the T41 site). Then, a high-fidelity DNA cloning kit (purchased from NEB, catalog number E5520S) was used, and the fragment between two digestion sites Xbal and Xhol of the NB1S3-WT plasmid was respectively replaced for the obtained mutant gene according to operations in the instruction, so as to construct seven expression plasmids NB1S3-K35, NB1S3-T41, NB1S3-K43, NB1S3-Y45, NB1S3-E61, NB1S3-K64 and NB1S3-P65. The mutation was confirmed to be successful by sequencing.

4. Construction of rhIL-2 Expression Strain Subjected to Site-Directed Mutation

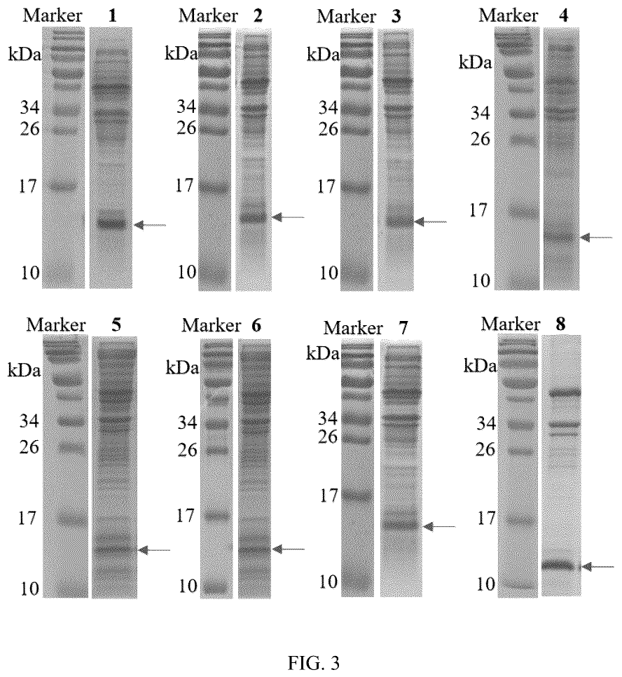

According to the structure of the plasmid pUltra recorded in the reference (Chatterjee, A. et al., Biochemistry, 52(10), 1828-1837, 2013), tRNA containing a carbonyl-terminal-containing lysine analogue which specifically recognized the structure shown in formula (I) of the present invention and tRNA synthetase encoding genes (a wild-type ancient Methanococcus pyrrole lysine synthetase encoding gene and an encoding gene corresponding to tRNA) and a chloramphenicol resistance gene (SEQ ID NO: 46) were obtained by full gene synthesis. Then, a DNA fragment (SEQ ID NO: 47) containing a CloDF13 replication initiation site was amplified from a commercial vector pCDF-duet1 by PCR amplification, and two DNA fragments were further sub-cloned and linked by using a high-fidelity DNA assembly cloning kit to obtain an helper plasmid NB1W (referring to FIG. 2, the plasmid was hereinafter referred to as the helper plasmid). A screening marker of this plasmid was chloramphenicol resistance. The helper plasmid and the expression plasmid (spectinomycin resistance) obtained in the step 3 were jointly transformed into Escherichia coli BL21(DE3) respectively, and double-positive strains (the double-positive strains referred to strains obtaining spectinomycin resistance and chloramphenicol resistance at the same time) were screened by spectinomycin resistance and chloramphenicol resistance plates: rhIL2-K35-BL21, rhIL2-T41-BL21, rhIL2-K43-BL21, rhIL2-Y45-BL21, rhIL2-E61-BL21, rhIL2-K64-BL21, rhIL2-P65-BL21.

The DNA fragment (SEQ ID NO: 46) containing the wild-type ancient Methanococcus pyrrole lysine synthetase encoding gene, the encoding gene corresponding to tRNA and the chloramphenicol resistance gene was as follows:

CCTTATGCGACTCCCTGCATTAGGGAGCTGTTGACAATTAATCAT
CGGCTCGTATAATGTGTGGAATTGTGAGCGGATAACAATTTCACA
AAGGAGGTCATATGGATAAAAAGCCTCTGAACACTCTGATTTCTG
CGACCGGTCTGTGGATGTCCCGCACCGGCACCATCCACAAAATCA
AACACCATGAAGTTAGCCGTTCCAAAATCTACATTGAAATGGCTT
GCGGCGATCACCTGGTTGTCAACAACTCCCGTTCTTCTCGTACCG
CTCGCGCACTGCGCCACCACAAATATCGCAAAACCTGCAAACGTT
GCCGTGTTAGCGATGAAGATCTGAACAAATTCCTGACCAAAGCTA
ACGAGGATCAGACCTCCGTAAAAGTGAAGGTAGTAAGCGCTCCGA
CCCGTACTAAAAAGGCTATGCCAAAAAGCGTGGCCCGTGCCCCGA
AACCTCTGGAAAACACCGAGGCGGCTCAGGCTCAACCATCCGGTT
CTAAATTTTCTCCGGCGATCCCAGTGTCCACCCAAGAATCTGTTT
CCGTACCAGCAAGCGTGTCTACCAGCATTAGCAGCATTTCTACCG
GTGCTACCGCTTCTGCGCTGGTAAAAGGTAACACTAACCCGATTA
CTAGCATGTCTGCACCGGTACAGGCAAGCGCCCCAGCTCTGACTA
AATCCCAGACGGACCGTCTGGAGGTGCTGCTGAACCCAAAGGATG
AAATCTCTCTGAACAGCGGCAAGCCTTTCCGTGAGCTGGAAAGCG
AGCTGCTGTCTCGTCGTAAAAAGGATCTGCAACAGATCTACGCTG
AGGAACGCGAGAACTATCTGGGTAAGCTGGAGCGCGAAATTACTC
GCTTCTTCGTGGATCGCGGTTTCCTGGAGATCAAATCTCCGATTC
TGATTCCGCTGGAATACATTGAACGTATGGGCATCGATAATGATA
CCGAACTGTCTAAACAGATCTTCCGTGTGGATAAAAACTTCTGTC
TGCGTCCGATGCTGGCCCCGAACCTGTACAACTATCTGCGTAAAC
TGGACCGTGCCCTGCCGGACCCGATCAAAATTTTCGAGATCGGTC
CTTGCTACCGTAAAGAGTCCGACGGTAAAGAGCACCTGGAAGAAT
TCACCATGCTGAACTTTTGCCAGATGGGTAGCGGTTGCACGCGTG
AAAACCTGGAATCCATTATCACCGACTTCCTGAATCACCTGGGTA
TCGATTTCAAAATTGTTGGTGACAGCTGTATGGTGTACGGCGATA
CGCTGGATGTTATGCACGGCGATCTGGAGCTGTCTTCCGCAGTAG
TGGGCCCAATCCCGCTGGATCGTGAGTGGGGTATCGACAAACCTT
GGATCGGTGCGGGTTTTGGTCTGGAGCGTCTGCTGAAAGTAAAAC
ACGACTTCAAGAACATCAAACGTGCTGCACGTTCCGAGTCCTATT
ACAATGGTATTTCTACTAACCTGTAACTAGTGTCTCCAGCTTGGC
TGTTTTGGCGGATGAGAGAAGATTTTCAGCCTGATACAGATTAAA
TCAGAACGCAGAAGCGGTCTGATAAAACAGAATTTGCCTGGCGGC
AGTAGCGCGGTGGTCCCACCTGACCCCATGCCGAACTCAGAAGTG
AGCACTGCAGCGGCTAACTAAGCGGCCTGCTGACTTTCTCGCCGA
TCAAAAGGCATTTTGCTATTAAGGGATTGACGAGGGCGTATCTGC
GCAGTAAGATGCGCCCCGCATTGGAAACCTGATCATGTAGATCGA
ATGGACTCTAAATCCGTTCAGCCGGGTTAGATTCCCGGGGTTTCC
GCCAAATTCGAAAAGCCTGCTCAACGAGCAGGCTTTTTTGCATCT
CGAGCAGCTCAGGGTCGAATTTGCTTTCGAATTTCTGCCATTCAT
CCGCTTATTATCACTTATTCAGGCGTAGCAACCAGGCGTTTAAGG
GCACCAATAACTGCCTTAAAAAAATTACGCCCCGCCCTGCCACTC
ATCGCAGTACTGTTGTAATTCATTAAGCATTCTGCCGACATGGAA
GCCATCACAAACGGCATGATGAACCTGAATCGCCAGCGGCATCAG
CACCTTGTCGCCTTGCGTATAATATTTGCCCATGGTGAAAACGGG
GGCGAAGAAGTIGTCCATATTGGCCACGTTTAAATCAAAACTGGT
GAAACTCACCCAGGGATTGGCTGAGACGAAAAACATATTCTCAAT
AAACCCTTTAGGGAAATAGGCCAGGTTTTCACCGTAACACGCCAC
ATCTTGCGAATATATGTGTAGAAACTGCCGGAAATCGTCGTGGTA
TTCACTCCAGAGCGATGAAAACGTTTCAGTTTGCTCATGGAAAAC
GGTGTAACAAGGGTGAACACTATCCCATATCACCAGCTCACCGTC
TTTCATTGCCATACGGAATTCCGGATGAGCATTCATCAGGCGGGC
AAGAATGTGAATAAAGGCCGGATAAAACTTGTGCTTATTTTTCTT
TACGGTCTTTAAAAAGGCCGTAATATCCAGCTGAACGGTCTGGTT
ATAGGTACATTGAGCAACTGACTGAAATGCCTCAAAATGTTCTTT
ACGATGCCATTGGGATATATCAACGGTGGTATATCCAGTGATTTT
TTTCTCCATTTTAGCTTCCTTAGCTCCTGAAAATCTCGATAACTC
AAAAAATACGCCCGGTAGTGATCTTATTTCATTATGGTGAAAGTT
GGAACCTCTTACGTGCCGATCAACGTCTCATTTTCGCCAAAAGTT
GGCCCAGGGCTTCCCGGTATCAACAGGGACACCAGGATTTATTTA
TTCTGCGAAGTGATCTTCCGTCACAGGTATTTATTCGGCGCAAAG
TGCGTCGGGTGATGCTGCCAACTTACTGATTTAGTGTATGATGGT
GTTTTTGAGGTGCTCCAGTGGCTTCTGTTTCTATCAGCTGTCCCT
CCTGTTCAGCTACTGACGGGGTGGTGCGTAACGGCAAAAGCACCG
CCGGACATCAGCGCGCGCTGCGGACAC

The DNA fragment (SEQ ID NO: 47) of the CloDF13 replication initiation site was as follows:

CGGACATCAGCGCGCGCTGCGGACACATACAAAGTTACCCACAGA
TTCCGTGGATAAGCAGGGGACTAACATGTGAGGCAAAACAGCAGG
GCCGCGCCGGTGGCGTTTTTCCATAGGCTCCGCCCTCCTGCCAGA
GTTCACATAAACAGACGCTTTTCCGGTGCATCTGTGGGAGCCGTG
AGGCTCAACCATGAATCTGACAGTACGGGCGAAACCCGACAGGAC
TTAAAGATCCCCACCGTTTCCGGCGGGTCGCTCCCTCTTGCGCTC
TCCTGTTCCGACCCTGCCGTTTACCGGATACCTGTTCCGCCTTTC
TCCCTTACGGGAAGTGTGGCGCTTTCTCATAGCTCACACACTGGT
ATCTCGGCTCGGTGTAGGTCGTTCGCTCCAAGCTGGGCTGTAAGC
AAGAACTCCCCGTTCAGCCCGACTGCTGCGCCTTATCCGGTAACT
GTTCACTTGAGTCCAACCCGGAAAAGCACGGTAAAACGCCACTGG
CAGCAGCCATTGGTAACTGGGAGTTCGCAGAGGATTTGTTTAGCT
AAACACGCGGTTGCTCTTGAAGTGTGCGCCAAAGTCCGGCTACAC
TGGAAGGACAGATTTGGTTGCTGTGCTCTGCGAAAGCCAGTTACC
ACGGTTAAGCAGTTCCCCAACTGACTTAACCTTCGATCAAACCAC
CTCCCCAGGTGGTTTTTTCGTTTACAGGGCAAAAGATTACGCGCA
GAAAAAAAGGATCTCAAGAAGATCCTTTGATCTTTTCTACTGAAC
CGCTCTAGATTTCAGTGCAATTTATCTCTTCAAATGTAGCACCTG
AAGTCAGCCCCATACGATATAAGTTGTAATTCTCATGTTAGTCAT
GCCCCGCGCCCACCGGAAGGAGCTGACTGGGTTGAAGGCTCTCAA
GGGCATCGGTCGAGATCCCGGTGCCTAATGAGTGAGCTAACTTAC
ATTAATTGCGTTGCGCTCACTGCCCGCTTTCCAGTCGGGAAACCT
GTCGTGCCAGCTGCATTAATGAATCGGCCAACGCGCGGGGAGAGG
CGGTTTGCGTATTGGGCGCCAGGGTGGTTTTTCTTTTCACCAGTG
AGACGGGCAACAGCTGATTGCCCTTCACCGCCTGGCCCTGAGAGA
GTTGCAGCAAGCGGTCCACGCTGGTTTGCCCCAGCAGGCGAAAAT
CCTGTTTGATGGTGGTTAACGGCGGGATATAACATGAGCTGTCTT
CGGTATCGTCGTATCCCACTACCGAGATGTCCGCACCAACGCGCA
GCCCGGACTCGGTAATGGCGCGCATTGCGCCCAGCGCCATCTGAT
CGTTGGCAACCAGCATCGCAGTGGGAACGATGCCCTCATTCAGCA
TTTGCATGGTTTGTTGAAAACCGGACATGGCACTCCAGTCGCCTT
CCCGTTCCGCTATCGGCTGAATTTGATTGCGAGTGAGATATTTAT
GCCAGCCAGCCAGACGCAGACGCGCCGAGACAGAACTTAATGGGC
CCGCTAACAGCGCGATTTGCTGGTGACCCAATGCGACCAGATGCT
CCACGCCCAGTCGCGTACCGTCTTCATGGGAGAAAATAATACTGT
TGATGGGTGTCTGGTCAGAGACATCAAGAAATAACGCCGGAACAT
TAGTGCAGGCAGCTTCCACAGCAATGGCATCCTGGTCATCCAGCG
GATAGTTAATGATCAGCCCACTGACGCGTTGCGCGAGAAGATTGT
GCACCGCCGCTTTACAGGCTTCGACGCCGCTTCGTTCTACCATCG
ACACCACCACGCTGGCACCCAGTTGATCGGCGCGAGATTTAATCG
CCGCGACAATTTGCGACGGCGCGTGCAGGGCCAGACTGGAGGTGG
CAACGCCAATCAGCAACGACTGTTTGCCCGCCAGTTGTTGTGCCA
CGCGGTTGGGAATGTAATTCAGCTCCGCCATCGCCGCTTCCACTT
TTTCCCGCGTTTTCGCAGAAACGTGGCTGGCCTGGTTCACCACGC
GGGAAACGGTCTGATAAGAGACACCGGCATACTCTGCGACATCGT
ATAACGTTACTGGTTTCACATTCACCACCCTGAATTGACTCTCTT
CCGGGCGCTATCATGCCATACCGCGAAAGGTTTTGCGCCATTCGA
TGGTGTCCGGGATCTCGACGCTCTCCCTTATGCGACTC

### Example 2 Expression and Purification of rhIL-2 Inserted with Unnatural Amino Acid Subjected to Site-Directed Mutation

1. Expression of Mutant rhIL-2 Incorporating with Unnatural Amino Acid

Seven expression strains rhIL2-K35-BL21, rhIL2-T41-BL21, rhIL2-K43-BL21, rhIL2-Y45-BL21, rhIL2-E61-BL21, rhIL2-K64-BL21 and rhIL2-P65-BL21 obtained in Example 1 were respectively inoculated into a LB medium (5 g/L yeast extract, 10 g/L tryptone and 10 g/L NaCl, containing 100 mg/L spectinomycin and 37.5 mg/L chloramphenicol), cultured at 37° C. for 5 hours to 8 hours, and then subjected to secondary expanded inoculation (in a medium with the same composition as the previous medium) until OD₆₀₀ of the bacterial solution was 2.0±0.2 to obtain a secondary seed solution.

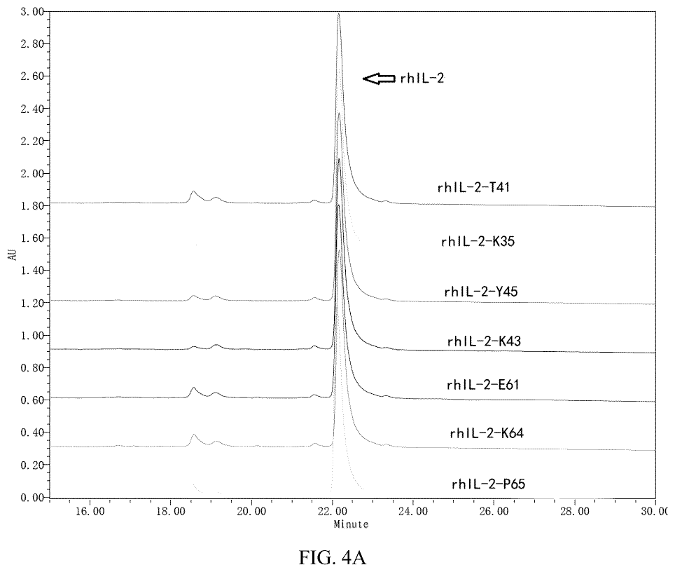

The above secondary seed solution was inoculated into a fermentation medium for fermentation culture in a 5 L fermentor. The culture volume was 2 L, the medium was 2×YT medium (16 g/L yeast extract, 10 g/L tryptone and 5 g/L NaCl), and the inoculation amount was 5% (v/v); the culture temperature was 37° C.; the pH value was controlled to be 6.90±0.05, and ammonia water or H₃PO₄ was automatically added when necessary; DO was controlled to be 30%, and DO was related to the rotating speed. When OD₆₀₀ of the bacterial solution reached 20.0±2.0, IPTG and the unnatural amino acid NOPK obtained in Preparation Example 3 were added, with final concentrations of 1 mM. Meanwhile, 50% glycerol started to be fed at a feeding speed of 0.6±0.1 mL/min. After inducing expression for 5 hours to 6 hours, bacterial cells were collected. SDS-PAGE electropherograms of various strains were shown in FIG. 3.

2. Isolation and Extraction of Mutant rhIL-2

The collected bacterial cells above were respectively suspended with a buffer solution (25 mM Tris, 6 mM EDTA and 1 mM DTT, at pH 8.0), added with 1% DNase (1 mg/mL) and 0.5% PMSF, mixed evenly, and homogenized for 3 times with an ultrahigh-pressure homogenizer under a pressure of 50 MPa to 80 MPa. The homogenate was centrifuged at 10,000 rpm for 20 minutes, and the lower crude inclusion bodies were collected.

The obtained crude inclusion bodies were washed twice with a washing buffer (20 mM Tris-HCl, 100 mM NaCl and 2% TritonX-100, at pH 8.0), and then washed once with ultrapure water to obtain purified inclusion bodies.

The purified inclusion bodies were dissolved with a denaturation buffer (20 mM Tris-HCl, 100 mM NaCl, 6 M guanidine hydrochloride and 1 mM DTT, at pH 8.0), and centrifuged at 10,000 rpm 30 minutes later, and the supernatant was collected, which was a denatured protein solution. A renaturation buffer (20 mM Tris-HCl and 100 mM NaCl, at pH 8.0) in a volume 4 times of the volume of the solution was added into the collected denatured protein solution, fully stirred and then allowed to stand for 12 hours, and centrifuged at 10,000 rpm to collect the supernatant, which was a renaturated protein solution.

The renaturated protein solution was concentrated to ¼ of the original volume with an ultrafiltration cassette (Millipore, Biomax-5) with a molecular weight cut-off of 5 kDa, the medium was substituted with a substitution buffer (20 mM Tris-HCl, at pH 8.0) until the conductivity was about 2 ms/cm, the solution was further concentrated until the protein concentration was about 0.5 mg/mL to 1 mg/mL, and centrifuged at 10,000 rpm, and then the supernatant was collected, so that crude proteins of mutant rhIL-2 were obtained, including rhIL2-K35, rhIL2-T41, rhIL2-K43, rhIL2-Y45, rhIL2-E61, rhIL2-K64 and rhIL2-P65, and could be directly used for subsequent PEG coupling.

### Example 3 Site-Directed Coupling Between PEG and rhIL-2 Inserted with Unnatural Amino Acid NOPK Subjected to Site-Directed Mutation

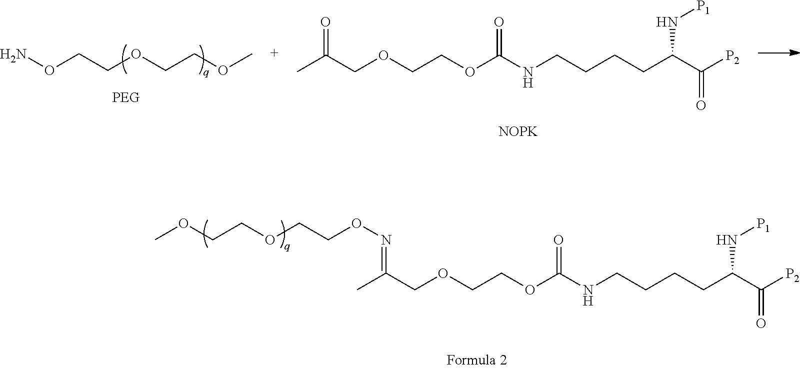

The site-directed coupling between the PEG and the rhIL-2 inserted with the unnatural amino acid subjected to site-directed mutation was shown in the synthetic route of Formula 2 (wherein the direction from of P₁ to P₂ was the direction from the N-terminal to the C-terminal of the amino acid sequence).

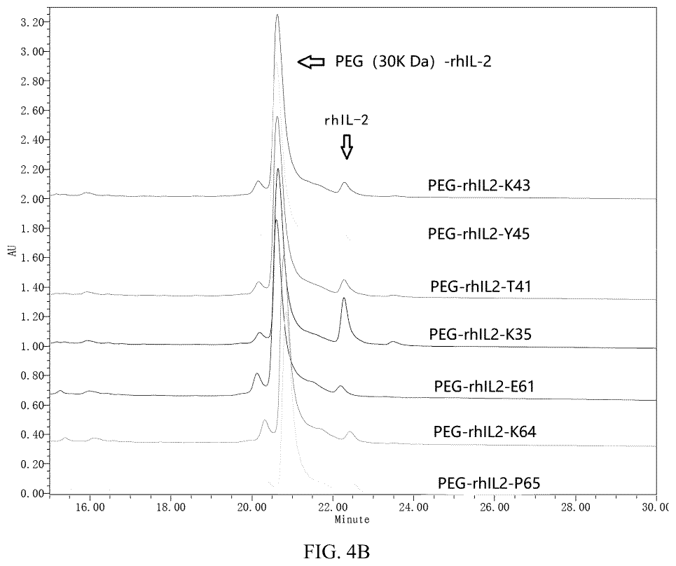

Taking the oximation reaction of 30 KD aminoxy PEG (i.e. hydroxyamino PEG) coupled with the rhIL-2 as an example, the coupling reaction was operated as follows: before the coupling reaction, the obtained target protein above was adjusted with 2 M acetic acid solution until the pH value was 4.0, adjusted with 20 mM sodium acetate buffer (at pH 4.0) until the protein concentration was about 1 mg/ml, added with a 30 KD aminoxy PEG solid (purchased from Beijing Jenkem Technology Co., Ltd.) according to 1:15 (a molar ratio of the protein to the aminoxy PEG), and fully shaken for dissolution to obtain a clear and transparent solution. Subsequently, the reaction solution was sealed, and shaken in a constant temperature shaker (25° C., 100 rpm) for reaction. The coupling situation was analyzed by RP-HPLC 48 hours later, as shown in FIG. 4A and FIG. 4B. Results showed that there was only a weak peak of rhIL-2 in FIG. 4B, which indicated that the seven target proteins were all coupled with the PEG, and coupling rates (100%—residual rhIL-2 concentration after coupling/rhIL-2 concentration when the coupling reaction time was zero×00%) were all greater than 95%, thus further indicating that the unnatural amino acid NBOK above was inserted into the target protein.

The mutant rhIL-2 proteins coupled with the PEG were respectively named as: 30 KD PEG-rhIL2-K35, 30 KD PEG-rhIL2-T41, 30 KD PEG-rhIL2-K43, 30 KD PEG-rhIL2-Y45, 30 KD PEG-rhIL2-E61, 30 KD PEG-rhIL2-K64 and 30 KD PEG-rhIL2-P65.
RP-HPLC analysis conditions were as follows:
mobile phase A (0.1% TFA-H₂O); and
mobile phase B (0.1% TFA-ACN).

| Chromatographic column | ZORBAX 300SB-C8, 4.6 × 150 mm, 5 μm | | |
|---|---|---|---|
| Detection wavelength | 214 nm | | |
| Temperature of column | 30° C. | | |
| thermerstat | | | |
| Temperature of sample | 15° C. | | |
| injector | | | |
| Flow velocity | 1.0 mL/min | | |
| Gradient elution | Time (min) | A (%) | B (%) |
| | 0 | 90 | 10 |
| | 10 | 65 | 30 |
| | 30 | 20 | 80 |
| | 31 | 90 | 10 |
| | 45 | 90 | 10 |

### Example 4 Purification of 30 KD PEG-rhIL2 Subjected to Site-Directed Modification

Chromatographic medium: Capto MMC; balance buffer: 20 mM sodium citrate buffer (pH=3.0), and elution buffer: 20 mM sodium citrate buffer—1 M NaCl (pH=7.8).

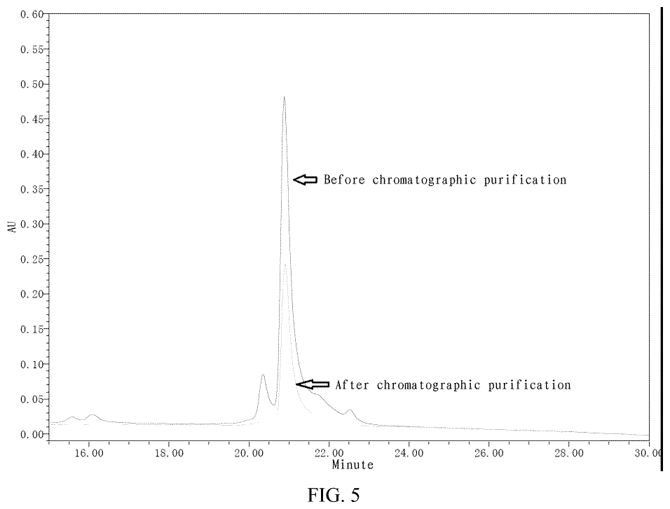

The purification process specifically included: adjusting 30 KD PEG-rhIL2-K35, 30 KD PEG-rhIL2-T41, 30 KD PEG-rhIL2-K43, 30 KD PEG-rhIL2-Y45, 30 KD PEG-rhIL2-E61, 30 KD PEG-rhIL2-K64 and 30 KD PEG-rhIL2-P65 coupling reaction solutions obtained in Example 3 with balance buffer respectively until the pH value was 3.0±0.2, with the conductivity less than or equal to 5 ms/cm, loading samples to Capto MMC, carrying out linear elution with an elution buffer (0-100% eluent, 20 CV), and collecting target protein components, so as to obtain target protein samples with a purity of about 95%. Taking 30 KD PEG-rhIL2-Y45 as an example, typical RP-HPLC profile of conjugate 30 KD PEG-rhIL2-Y45 after purification and rhIL2-Y45 before coupling were shown in FIG. 5. Other protein samples had similar purification results.

### Example 5 In-Vitro Activity Evaluation on 30 KD PEG-rhIL2 Subjected to Site-Directed Modification (STAT5 Phosphorylation Assay)

In this method, two cell lines were adopted, wherein mouse CTLL-2 cells were a cell line containing IL-2Rαβγ, and human YT cells were a cell line containing IL-2Rβγ, and rhIL-2 activated the JAK-STAT signal pathway by binding with IL-2R on the cell surface. Different modification sites of various samples had different relative activities to the two kinds of cells. The lower the percentage change of EC50 ratio of YT cells/CTLL-2 cells was, the better the effect of promoting the immune function of the samples was, and on the contrary, the better the effect of inhibiting the immune function was.

The specific process was as follows: the mouse CTLL-2 cells (purchased from American Type Culture Collection) and the human YT cells were cultured in their own media (CTLL-2 cell medium: RPMI 1640+10% FBS+400 IU/mL rhIL-2, 2 mM L-glutamine and 1 mM sodium pyruvate; and YT cell medium: RPMI 1640+10% FBS+1 mm Non-Essential Amino Acids Solution (purchased from Gibco, product catalog number 11140050)) to a sufficient amount at condition of 37° C. and 5% carbon dioxide, and starved for 4 hours before detection, and then the cell density was adjusted to be 1×10⁶ cells/mL for later use. 30 KD PEG-rhIL2-K35, 30 KD PEG-rhIL2-T41, 30 KD PEG-rhIL2-Y45, 30 KD PEG-rhIL2-E61 and 30 KD PEG-rhIL2-P65, samples of rhIL2-K35, rhIL2-T41, rhIL2-Y45, rhIL2-E61 and rhIL2-P65 before coupling, and a reference rhIL-2 (purchased from Beijing Solarbio Science & Technology Co., Ltd., catalog number: P00020) were gradiently diluted respectively, and each sample had a total of six concentrations (in the CTLL-2 cell assay, the reference had a concentration range of 0.004 ng/mL to 4 ng/mL, and was subjected to 4-fold gradient dilution; in the YT cell assay, the reference had a concentration range of 2.1 ng/mL to 510 ng/mL, and was subjected to 3-fold gradient dilution; and other samples had a concentration range obtained through pre-experimental screening, which corresponded to corresponding EC50 values in Table 2). The cells were stimulated at 37° C. for 10 minutes and then lysed, a western blot experiment was carried out, the cells were hybridized with a pSTAT5 antibody (purchased from CST, product catalog number 9359L) and β-actin (purchased from CST, product catalog number 8457S), protein amounts of pSTAT5 and β-actin in the cell lysate were detected, and EC50 was calculated according to gray scale results of pSTAT5/β-actin and sample concentrations. Results were shown in Table 2. The results showed that rhIL2 inserted with unnatural amino acids NOPK at the K35 site, the T41 site, the Y45 site, the E61 site and the P65 site and coupled with 30 KD PEG all met original design requirements (compared with the reference, the percentage change of EC50 ratio was lower than that of the reference).

TABLE 2
Results of STAT5 phosphorylation assay

| | | | | | | Percentage change |
|---|---|---|---|---|---|---|
| STAT5 phosphorylation assay | | | STAT5 phosphorylation assay | | | of EC50 ratio |
| (CTLL-2 cells) | | | (YT cells) | | | of YT/CTLL-2 |
| | EC50 (ng/ml) | | | EC50 (ng/ml) | | cells of 30 KD |
| | | 30 KD | Mutation | | 30 KD | PEG-rhIL2 |
| Mutation site | rhIL-2 | PEG-rhIL2 | site | rhIL-2 | PEG-rhIL2 | sample* |
| Reference | 0.12 | / | Reference | 21.3 | / | 100% |
| K35 | 0.13 | 2.27 | K35 | 52.7 | 213 | 53% |
| T41 | 0.16 | 4.41 | T41 | 59.9 | 340 | 43% |
| Y45 | 1.52 | 19.01 | Y45 | 147.0 | 218 | 6% |
| E61 | 0.17 | 4.23 | E61 | 96.1 | 667 | 89% |
| P65 | 0.06 | 4.15 | P65 | 84.0 | 672 | 91% |

*Calculation formula: [EC50 of YT cells of 30 KD PEG-rhIL2 sample/EC50 of CTLL-2 cells of 30 KD PEG-rhIL2 sample]/[EC50 of YT cells of reference/EC50 of CTLL-2 cells of reference] × 100%

### Example 6 In-Vivo Pharmacokinetic Study in Mouse

Female C57 mice (SPF grade, purchased from Zhejiang Vital River Laboratory Animal Technology Co., Ltd.) were used in the experiment, Quanqi rhIL-2 (purchased from Shandong Quangang Pharmaceutical Co., Ltd.) was used as a positive control drug, and the metabolism situation of 30 KD PEG-rhIL2-Y45 in the mice was investigated. The 30 KD PEG-rhIL2-Y45 and the Quanqi rhIL-2 were respectively administrated by single intravenous injection according to 1 mg/kg, and blood sampling was carried out according to the following blood sampling points, with 0.5 mL (n=5) for each point: before administration, and 0.0833 hour, 0.5 hour, 1 hour, 4 hours, 8 hours, 16 hours and 24 hours after administration. In addition, additional 5 blood sampling points were set for the 30 KD PEG-rhIL2-Y45: 48 hours, 72 hours, 96 hours, 120 hours and 144 hours. The blood sample was placed at room temperature for 15 minutes and centrifuged at 6,800 g/min for 6 minutes to obtain serum. The plasma concentration in the serum was analyzed by the following method:

(1) Coating: 50 μL of 1 μg/mL working solution of Anti-IL-2 antibody (purchased from abcam, product catalog number: ab9618) was added into each well in a high-adsorption 96-well plate, and incubated at 2° C. to 8° C. overnight. (2) Washing: the liquid in the well was discarded, and the well was washed with 1 xPBST (0.05% Tween-20) for 3 times, according to 300 μL/well. (3) Sealing: a casein sealing solution (purchased from Thermo, product catalog number 37528) was added according to 200 μL/well, and allowed to stand at room temperature for 90 minutes. (4) Washing: the liquid in the well was discarded, and the well was washed with 1 xPBST for 3 times, according to 300 μL/well. (5) Sample loading: the Quanqi rhIL-2, the 30 KD PEG-rhIL2-Y45 and the serum sample to be tested were gradiently diluted with mouse serum, transferred to a micro-well plate according to 50 μL/well, and allowed to stand at room temperature for 120 minutes. (6) Washing: the liquid in the well was discarded, and the well was washed with 1×PBST for 3 times, according to 300 μL/well. (7) Primary antibody: 50 μL of 0.25 μg/mL working solution of IL-2 Monoclonal Antibody (BG5) and Biotin (purchased from Invitrogen, product catalog number M600B) was added into each well, and allowed to stand at room temperature for 60 minutes. (8) Washing: the liquid in the well was discarded, and the well was washed with 1×PBST for 3 times, according to 300 μL/well. (9) Secondary antibody: Pierce™ High Sensitivity Streptavidin-HRP (purchased from Thermo, product catalog number 21130) was diluted for 4,000 times with a casein sealing solution, and 50 μL was added into each well, and allowed to stand at room temperature for 60 minutes. (10) Washing: the liquid in the well was discarded, and the well was washed with 1×PBST for 4 times, according to 300 μL/well. (11) Substrate: 50 μL of 1-Step™ Turbo TMB-ELISA Substrate Solution (purchased from Thermo, product catalog number 34022) was added into each well. (12) Stopping and reading: 2 M sulfuric acid stop solution was added 25 minutes later, and light absorption values at 450 nm and 650 nm were read on a microplate reader (purchased from Perkin Elmer, model EnSight). (13) Analysis: four-parameter fitting was carried out by Dazdaq Ltd. WorkOut 1.5 analysis software, and concentrations and units corresponding to the Quanqi rhIL-2 and the 30 KD PEG-rhIL2-Y45 were input to fit a curve for each of the two, so as to respectively calculate the plasma concentration in the serum to be tested. According to the non-compartment model (statistical moment parameter) of DAS software, average half-life t_{1/2} of Quanqi rhIL-2 and 30 KD PEG-rhIL2-Y45 were calculated to be 0.77 hour and 14.69 hours respectively. The pharmacokinetic parameters of Quanqi rhIL-2 and 30 KD PEG-rhIL2-Y45 were shown in Table 3.

TABLE 3
Pharmacokinetic parameters (n =
5) of Quanqi rhIL-2 and 30KD PEG-rhIL2-Y45

| | Pharmacokinetic parameter (n = 5) | | | |
|---|---|---|---|---|
| | Quanqi rhIL-2 | | 30KD PEG-rhIL2-Y45 | |
| | Average | Standard | Average | Standard |
| Parameter | value | deviation | value | deviation |
| AUC₀₋ₜ (ng/mL × h) | 2445 | 133 | 168141 | 7052 |
| MRT₀₋ₜ (h) | 0.41 | 0.035 | 9.42 | 0.361 |
| t_{1/2} (h) | 0.77 | 0.142 | 14.69 | 3.051 |
| Tₘₐₓ (h) | 0.083 | 0.000 | 0.361 | 0.241 |
| CL (L/h/kg) | 0.409 | 0.022 | 0.006 | 0.000 |
| Vz (L/kg) | 0.455 | 0.077 | 0.126 | 0.024 |
| Cₘₐₓ (ng/mL) | 6197 | 547 | 26939 | 2219 |

### Example 7 In-Vivo Pharmacodynamical Study in Mouse—Anti-Tumor Activity

Female Balb/c mice (SPF grade, Zhejiang Vital River Laboratory Animal Technology Co., Ltd.) were used in the experiment. A cell suspension of 2×10⁵/0.1 mL/mouse CT26.WT (purchased from ATAC, product catalog number CRL-2638) and a cell suspension of 4×10⁵/0.1 mL/mouse H22 (purchased from CCTCC, product catalog number GD00091) were subcutaneously inoculated into right backs of the mice. When the tumor volume reached about 50 mm³, the mice were randomly grouped, with seven mice in each group, and respectively administrated with vehicle (1 xPBS), 0.7 mg/kg 30 KD PEG-rhL2-T41, 5.0 mg/kg 30 KD PEG-rhIL2-T41, 0.7 mg/kg 30 KD PEG-rhIL2-Y45 and 5.0 mg/kg 30 KD PEG-rhIL2-Y45 (with administration volumes of 10 mL/kg). During the experiment, weights and tumor volumes of the animals were measured 3 times every week, and administration modes and experimental results were shown in Table 4. Calculation formula of relative inhibition rate TGI_{TW} (%): (T_{WC}−T_{WT})/T_{WC}×100%, wherein Tw was the average tumor weight of the vehicle control group, and T_{WT} was the average tumor weight of the treatment group.

Results showed that, compared with the vehicle group, high-dosage and low-dosage groups of the two test substances had significant inhibitory effects on homograft tumors of mouse colon cancer CT26.WT and mouse liver cancer H22.

TABLE 4
Administration modes of test substances in homograft tumor models of CT26.WT
and H22, and influences of test substances on tumor weights of animals

| | | | | | CT26.WT model | | H22 model | |
|---|---|---|---|---|---|---|---|---|
| | | | | | Average | | Average | |
| | | Administration mode | | | value ± | | value ± | |
| | | Admin- | | | standard | | standard | |
| | | istration | Admin- | Admin- | error of | | error of | |
| | Test | dosage | istration | istration | tumor | TGI_{TW} | tumor | TGI_{TW} |
| Group | substance | (mg/kg) | route | frequency | weight (g) | (%) | weight (g) | (%) |
| 1 | Vehicle | N/A | IV | Q5D × 3 | 2.74 ± | / | 1.88 ± | / |
| | control | | | | 0.18 | | 0.11 | |
| 2 | High dosage | 5.0 | IV | Q5D × 3 | 0.32 ± | 88 | 0.38 ± | 80 |
| | of 30 KD | | | | 0.12 *** | | 0.08 *** | |
| | PEG-rhIL2- | | | | | | | |
| | T41 | | | | | | | |
| 3 | Low dosage | 0.7 | IV | Q5D × 3 | 0.64 ± | 77 | 0.69 ± | 63 |
| | of 30 KD | | | | 0.05 *** | | 0.05 *** | |
| | PEG-rhIL2- | | | | | | | |
| | T41 | | | | | | | |
| 4 | High dosage | 5.0 | IV | Q5D × 3 | 0.22 ± | 92 | 0.4 ± | 79 |
| | of 30 KD | | | | 0.09 | | 0.07 *** | |
| | PEG-rhIL2- | | | | | | | |
| | Y45 | | | | | | | |
| 5 | Low dosage | 0.7 | IV | Q5D × 3 | 0.93 ± | 66 | 0.82 ± | 57 |
| | of 30 KD | | | | 0.16 *** | | 0.15 *** | |
| | PEG-rhIL2- | | | | | | | |
| | Y45 | | | | | | | |

Note:
*: p < 0.05;
**: p < 0.01;
***: p < 0.001, compared with the vehicle group.

### Example 8 In-Vivo Pharmacodynamical Study in Mouse—Expression of Immune Cell Population

Female Balb/c mice (SP A grade, Zhejiang Vital River Laboratory Animal Technology Co., Ltd.) were used in the experiment. A cell suspension of 2×10⁵/0.1 mL/mouse CT26.WT was subcutaneously inoculated into right backs of the mice. When the tumor volume reached about 100 mm³, the mice were randomly grouped, with 3 mice in each group, and respectively administrated with various test substances and Quanqi rhIL-2 (purchased from Shandong Quangang Pharmaceutical Co., Ltd.) according to Table 5, with administration volumes of 10 mL/kg. On the 5^{th} day, tumor tissue samples of various groups were collected for flow cytometry detection of changes in cell populations of CD8⁺ T cells and CD4⁺ Treg cells. Results were shown in Table 5.

TABLE 5
Administration mode of test substances in homograft tumor model of CT26.WT,
and influence of test substances on immune cell population

| | | | | | Percentage of each cell | | |
|---|---|---|---|---|---|---|---|
| | | | | | population in leucocytes (%) | | |
| | | Admin- | | | | | CD8⁺ |
| | | istration | Admin- | Admin- | | | T cells/ |
| | Test | dosage | istration | istration | CD8⁺ | CD4⁺ | CD4⁺ |
| Group | substance | (mg/kg) | route | frequency | T cells | Treg | Treg |
| 1 | Vehicle | N/A | IV | Single | 3.3 | 1.5 | 2.2 |
| | control | | | dosage | | | |
| 2 | Quanqi | 2.0 | IV | QD × 5 | 8.5 | 6.7 | 1.3 |
| | rhIL2 | | | | | | |
| 3 | 30 KD | 2.0 | IV | Single | 36.0 | 1.3 | 27.7 |
| | PEG-rhIL2- | | | dosage | | | |
| | T41 | | | | | | |
| 4 | 30 KD | 2.0 | IV | Single | 30.1 | 1.3 | 23.2 |
| | PEG-rhIL2- | | | dosage | | | |
| | Y45 | | | | | | |

Compared with Quanqi rhIL2, the 30 KD PEG-rhIL2-T41 and the 30 KD PEG-rhIL2-Y45 had significantly increased proportions of CD8⁺ T cells, significantly decreased proportions of CD4⁺ Treg cells, and significantly increased proportions of CD8⁺ T/CD4⁺ Treg, showing excellent immune enhancement effects.

### Example 9 Reduction of Active Group of Lys-Azido

With reference to the method in Example 1, an expression strain of rhIL-2 (rhGH-V91 for short) in which the amino acid (valine) codons at the 91^{st} site were mutated into amber codons was constructed, and primers used in the construction process were as follows:

V91-F:
(SEQ ID NO: 48)
5′-GATTTCCAATATCAACTAGATTGTTCTGGAACTGA-3′
V91-R:
(SEQ ID NO: 49)
5′-TCAGTTCCAGAACAATCTAGTTGATATTGGAAATC-3′

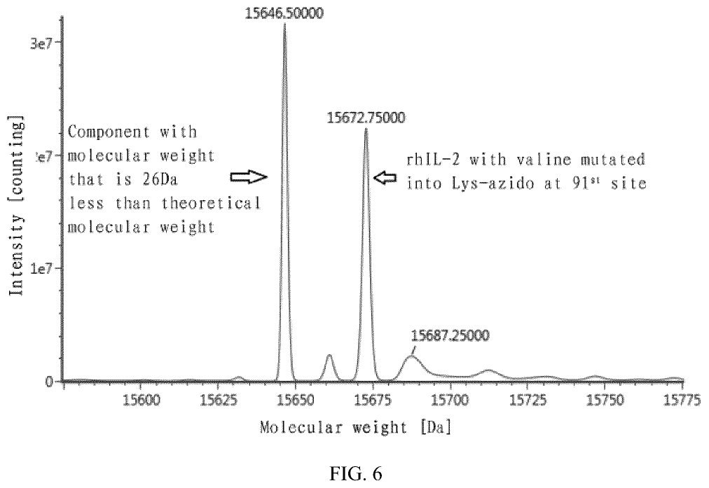

With reference to Example 2, the above rhGH-V91 expression strain was used to express the rhIL-2 subjected to mutation to Lys-azido at the 91^{st} site by adding Lys-azido in the fermentation process, and purification was carried out by the corresponding purification method in Example 2. The complete molecular weight of the rhIL-2 subjected to mutation to Lys-azido at the 91^{st} site was analyzed by liquid chromatography-mass spectrometry (high resolution mass spectrometer: XevoG2-XS Q-Tof, Waters Company; ultra-high performance liquid chromatography: UPLC (Acquity UPLC I-Class), Waters Company), as shown in FIG. 6. Results showed that there was a component which was about 26 Da smaller than the theoretical molecular weight (15672.75 Da) in the sample, and it was inferred that the azido structure (—N₃) at the terminal of Lys-azido was reduced to the product of (—NH₂).

The reduction of Lys-azido could lead to the failure to couple the rhIL-2 subjected to mutation to Lys-azido with BCN-PEG, thus reducing the coupling rate. However, when the rhIL-2 containing the unnatural amino acid of the present invention was coupled with PEG, the coupling rate was significantly increased, thus significantly improving the reaction efficiency.

Unless otherwise specified, the terms used in the present invention have the meanings commonly understood by those skilled in the art.

The described embodiments of the present invention are for exemplary purposes only, and are not intended to limit the protection scope of the present invention. Those skilled in the art can make various other substitutions, changes, and improvements within the scope of the present invention. Therefore, the present invention is not limited to the above embodiments, but only limited by the claims.

1. A human interleukin 2-polyethylene glycol conjugate, comprising a recombinant human interleukin 2 containing at least one unnatural amino acid and PEG coupled to the at least one unnatural amino acid;
the unnatural amino acid is a compound containing a carbonyl terminal group and having a structure as shown in formula (I) or an enantiomer thereof, and the PEG is coupled to the at least one unnatural amino acid by forming an oxime bond between the carbonyl terminal group and the PEG containing a hydroxylamine terminal group, 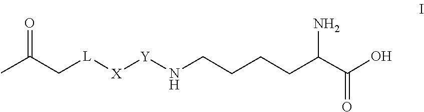
wherein X represents —O—, —S—, —NH— or —CH₂—, Y represents —O—, —S—, —C(O)—, —S(O)— or —CH₂—, and L represents substituted or unsubstituted C0-C20 linear or branched alkylene, one or more —CH₂— are optionally substituted by one or more of —O—, —S—, —NH—, —C(O)— and —S(O)—;
when X represents —O— and Y represents —C(O)—, L does not represent —CH₂CH₂—; and
when L represents a substituent, the substituent may be selected from one or more of hydroxyl, sulfhydryl, halogen, nitro, cyano, alkyl, alkenyl, alkynyl, alkoxy, acyl, acylamino, carboxyl, ester, amino, sulfonyl, sulfinyl, cycloalkyl, heterocyclyl, aryl and heteroaryl.

2. The human interleukin 2-polyethylene glycol conjugate according to claim 1, wherein the recombinant human interleukin 2 is a protein shown in SEQ ID NO: 3 or a functional active fragment thereof; and
preferably, in the recombinant human interleukin 2 containing at least one unnatural amino acid, a position of the at least one unnatural amino acid is selected from one or more sites corresponding to sites P34, K35, T37, R38, L40, T41, F42, K43, F44, Y45, E61, E62, K64, P65, E67, E68, N71, L72 and Y107 of SEQ ID NO: 2.

3. The human interleukin 2-polyethylene glycol conjugate according to claim 1 or 2, wherein the substituent is selected from one or more of hydroxyl, sulfhydryl, halogen, nitro, cyano, C1-C6 alkyl, C1-C6 alkoxy, acyl, acylamino, carboxyl, ester, amino, sulfonyl, sulfinyl, C3-C8 cycloalkyl, C3-C8 heterocyclyl, C6-C20 aryl and C4-C10 heteroaryl;
preferably, L represents C0-C10 linear or branched alkylene, wherein one or more —CH₂— are optionally substituted by —O—; and more preferably, L represents C0-C6 linear alkylene; and/or
X represents —O—, —S—, —NH— or —CH₂—; and/or
Y represents —C(O)—.

4. The human interleukin 2-polyethylene glycol conjugate according to any one of claims 1-3, wherein the unnatural amino acid is a compound with a structure as shown in formula (I-1), 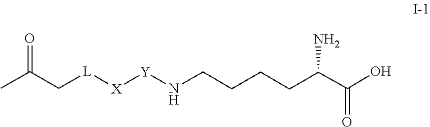
wherein X, Y and L are each independently as defined in any one of claims 1-3.

5. The human interleukin 2-polyethylene glycol conjugate according to claim 4, wherein the unnatural amino acid is a compound with a structure as shown in formula (I-2), formula (I-3), formula (I-4) or formula (I-5), 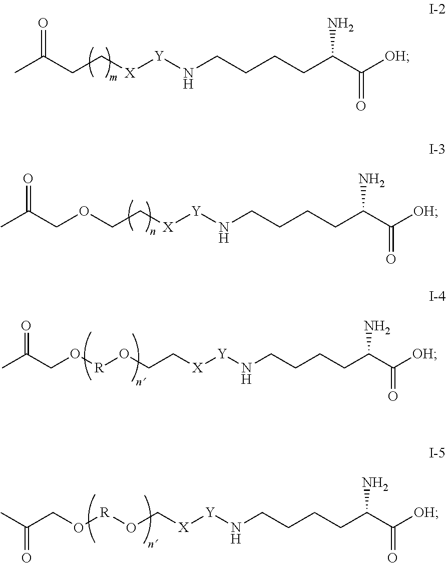
wherein X and Y are each independently as defined in any one of claims 1-4, m and n each independently represent an integer of 0 to 8, preferably represent an integer of 0 to 5, and more preferably represent 0, 1, 2 or 3;
when X represents —O— and Y represents —C(O)—, m does not represent 1; and
R represents C1-C4 linear or branched alkylene, and preferably represents —CH₂—, —CH₂CH₂— or —CH₂CH₂CH₂—; n′ represents an integer of 0 to 5, preferably represents an integer of 0 to 3, and more preferably represents 0, 1 or 2.

6. The human interleukin 2-polyethylene glycol conjugate according to claim 5, wherein the unnatural amino acid is a compound with one of the structures as follows,

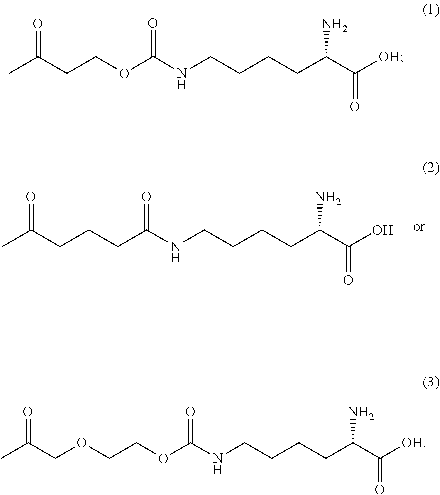 [SMILES: CC(=O)CCOC(=O)NCCCC[C@H](N)C(=O)O ; CC(=O)CCCC(=O)NCCCC[C@H](N)C(=O)O ; CC(=O)COCCOC(=O)NCCCC[C@H](N)C(=O)O]

7. The human interleukin 2-polyethylene glycol conjugate according to any one of claims 1-6, wherein the PEG containing a hydroxylamine terminal group has a molecular weight of 20 KD to 50 KD.

8. The human interleukin 2-polyethylene glycol conjugate according to any one of claims 1-7, wherein the recombinant human interleukin 2 containing at least one unnatural amino acid is prepared by a codon expansion technology or by means of chemical synthesis.

9. The human interleukin 2-polyethylene glycol conjugate according to claim 8, wherein the codon expansion technology is implemented in Escherichia coli.

10. Use of the human interleukin 2-polyethylene glycol conjugate according to any one of claims 1-9 in preparation of a drug for promoting immunity, preventing and/or treating solid tumors and hematologic tumors, and/or expanding CD8⁺ T cells;
preferably, the solid tumors refer to bladder cancer, bone cancer, brain cancer, breast cancer, colorectal cancer, esophageal cancer, ocular cancer, head and neck cancer, kidney cancer, lung cancer, melanoma, ovarian cancer, pancreatic cancer or prostate cancer; and
preferably, the hematologic tumors refer to chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B-cell lymphoma, nodal marginal zone B-cell lymphoma, Burkitt lymphoma, non-Burkitt high-grade B-cell lymphoma, primary mediastinal B-cell lymphoma (PMBL), immunoblastic large cell lymphoma, precursor B-cell lymphoblastic lymphoma, B-cell prolymphocytic leukemia, lymphplasmacytic lymphoma, splenic marginal zone lymphoma, plasma cell myeloma, plasmacytoma, mediastinal (thymic) large B-cell lymphoma, intravascular large B-cell lymphoma, primary effusion lymphoma, or lymphomatoid granulomatosis.
